(19) United States
(12) Patent Application Publication
(10) Pub. No.: US 2026/0284190 A1
(43) Pub. Date: Sep. 24, 2026

(54) METHODS FOR THE TREATMENT OF T-CELL ACUTE LYMPHOBLASTIC LEUKEMIA

(71) Applicants: Institut Curie, Paris (FR); CENTRE NATIONAL DE LA RECHERCHE SCIENTIFIQUE, Paris (FR); Institut National de la Sante et de la recherche Medicale, Paris (FR)

(72) Inventors: Jacques GHYSDAEL, Gif sur Yvette (FR); Christine TRAN QUANG, Villebon sur Yvette (FR)

(21) Appl. No.: 19/474,265

(22) PCT Filed: Apr. 13, 2024
(86) PCT No.: PCT/EP2024/060103

§ 371 Date: Oct. 10, 2025

(30) Foreign Application Priority Data
Apr. 13, 2023 (EP) 23305552.4

(51) Int. Cl. A61K 39/395; A61K 38/05; A61K 38/17; A61K 39/00; A61P 35/02

(52) U.S. Cl. CPC A61K 39/3955; A61K 38/05; A61K 38/1793; A61K 39/39591; A61P 35/02; A61K 2039/505

(57) ABSTRACT

The present invention relates to the combination of anti-CD3 agent, in particular monoclonal antibody, with immunotherapeutic agents, and its use in oncology, for treating T Cell Acute Lymphoblastic Leukemia (T-ALL). The invention also relates to a pharmaceutical composition comprising said combination and the use of said combination to induce cell death of T-ALL cells.

### FIELD OF THE INVENTION

The invention relates to the field of oncology, in particular to methods and pharmaceutical compositions for the treatment of T Cell Acute Lymphoblastic Leukemia (T-ALL).

### BACKGROUND OF THE INVENTION

Acute lymphoblastic leukemia (ALL) is the most frequent childhood cancer. Particularly, T cell lineage ALL (T-ALL), which represents 10% of pediatric and 25% of adult ALL cases, are of poorer prognosis and with relapse being of dismal prognosis (Inaba et al., 2020). T cell acute lymphoblastic leukemia (T-ALL) results from the leukemic transformation of T cell precursors and abnormal blockade in differentiation (Reviewed in Belver & Ferrando, 2016; Girardi et al., 2017, Cordo et al., 2020). Cytogenetic and global transcriptomic analyses allowed to classify T-ALL into molecular groups, characterized by abnormal expression of specific transcription factors (such as TAL; LMO1/2; TLX1/3; HOXA and others) and a block of T cell precursors differentiation at specific stages. The TAL/LMO group is the largest one and often corresponds to a maturation arrest at the double positive (DP) to single positive (SP) transition (TCR^{αβ+}, mature T-ALL). The maturation stage arrest of TLX1 and TLX3 T-ALL cells occurs at the cortical stage, preceding TCR^{αβ} expression (immature T-ALL) or often entails expression of TCRY^{γδ}.

In the last years, early T-cell precursor (ETP) T-ALL has been recognized as heterogeneous entities arrested at an early differentiation stage and harboring a signature of stem or early myeloid progenitors (reviewed in Girardi et al, 2017). Across these groups several additional and recurrent genetic abnormalities have been found, including loss of tumor suppressive genes (e.g., PTEN and CDKN2A) and activation of oncogenic pathways (e.g. NOTCH1, IL7R/JAK) (Girardi et al., 2017). Although recent immune-therapeutic approaches, including monoclonal antibodies (mAb), bispecific antibodies and chimeric antigen receptor (CAR)-T cells, have considerably diversified and improved the therapeutic arsenal for B-cell acute lymphoblastic leukemia, therapeutic advances are so far missing in T-ALL.

Searching for T-ALL new therapeutic strategies, the inventors have previously shown that treatment of TCR^{αβ+} and TCR^{γδ+} T-ALL patients-derived xenografts, which represents over 50% of T-ALL pediatric cases, with anti-CD3 monoclonal antibodies (human anti-CD3 mAbs), including the clinically relevant Teplizumab or Foralumab, results in leukemic cells apoptosis within hours, profound anti-leukemic effects over time, durable remission, and improved host survival (Trinquand et al., 2016; Tran Quang et al., 2020). The inventors also showed that these anti-CD3 monoclonal antibodies strongly cooperate with chemotherapy in T-ALL patients-derived xenografts (Tran Quang et al., 2020). Since teplizumab and foralumab, which are first-in-class human anti-CD3 mAb, both show only minor adverse effects in humans (for review Kuhn et al., 2016; Herold et al., 2019) and regarding the previous obtained encouraging results, these anti-CD3 monoclonal antibodies offer a novel immune-therapeutic option for the treatment of T-ALL.

However, monotherapy of mice carrying TCR⁺ T-ALL patients-derived xenografts (T-ALL PDX) with Teplizumab or Foralumab ultimately results in leukemia relapse and resistance to TCR-directed therapy. Thus, there is still a need to identify new treatments for T-ALL and improve leukemic cells vulnerability to anti-CD3 monoclonal antibodies therapy.

Here, to find alternative and effective curative options for T-ALL in the context of TCR-directed therapies and in particular overcome leukemia relapse and resistance, the inventors investigated specific combinations which would be effective in T-ALL. The inventors discovered that combination of anti-CD3 monoclonal antibodies with specific immunotherapeutic agents, like other specific monoclonal antibodies, allows T-ALL cells death and anti-leukemic effect in a synergistic manner. There is no disclosure in the art of such a combination of an anti-CD3 agent and at least one immunotherapeutic agent for treating T-ALL, particularly combination of an anti-CD3 monoclonal antibody and at least one immunotherapeutic agent for treating T-ALL.

### SUMMARY OF THE INVENTION

The invention relates to methods and pharmaceutical compositions for the treatment of T Cell Acute Lymphoblastic Leukemia (T-ALL). In particular, the invention relates to an anti-CD3 agent for use in combination with at least one immunotherapeutic agent in the treatment of T Cell Acute Lymphoblastic Leukemia (T-ALL) in a subject in need thereof.

According to other optional features of the anti-CD3 agent for use according to the invention, it can optionally include one or more of the following characteristics alone or in combination:
said anti-CD3 agent is a small organic molecule, a polypeptide, an aptamer, an oligonucleotide (such as an antisense oligonucleotide, a siRNA, a shRNA, a DNA aptamer, or an RNA aptamer), or an antibody;
said antibody is a monoclonal antibody (mAb) selected from the group comprising: foralumab, teplizumab, otelixizumab, visilizumab, blinatumomab, and catumaxomab or any combination thereof;
said immunotherapeutic agent is a monoclonal antibody or a Fc-fusion protein;
said immunotherapeutic agent is a monoclonal antibody targeting at least one target selected from the group consisting of: PD1, 4-1BB, OX40, PDL1, TRAIL, TNFR1, TNFR2, TNFα/LTα, CD30, CD38 and/or CTLA4;
said immunotherapeutic agent is selected from the group consisting of: an anti-4-1BB agonistic mAb such as urelumab or utomilumab; an anti-OX40 agonistic mAb such as MEDI6469 (9B12), MEDI0562, PF-04518600, INCAGN01949, BMS-986178, MOXR0916 (RG7888), GSK3174998 or MEDI6383; an anti-PD1 antagonistic mAb such as pembrolizumab, nivolumab, cemiplimab, dostarlimab or pidilizumab (CT-011); an anti-PDL1 antagonistic mAb such as atezolizumab, avelumab or durvalumab; anti-TRAIL agonistic mAb; anti-TNFR1 antagonistic mAb; anti-TNFR2 antagonistic mAb; an anti-TNFα/LTα antagonistic mAb such as infliximab, adalimumab, certolizumab or golimumab; an anti-CD30 mAb such as brentuximab; an anti-CD38 mAb such as daratumumab; and an anti-CTLA4 mAb such as ipilimumab or tremelimumab, or any combination thereof;
said immunotherapeutic agent is a combination of at least two immunotherapeutic agents selected from an anti-4-1BB agonistic mAb, an anti-PD1 antagonistic mAb, an anti-PDL1 antagonistic mAb, an anti-OX40 agonistic mAb, and an anti-TNFα/LTα antagonistic mAb;
said immunotherapeutic agent is a combination of at least one an anti-4-1BB agonistic mAb and at least one anti-PD1 antagonistic mAb.
said immunotherapeutic agent is a combination of an anti-4-1BB agonistic mAb and an anti-OX40 agonistic mAb.
said immunotherapeutic agent is a combination of at least one anti-4-1BB agonistic mAb, at least one anti-OX40 agonistic mAb and at least one anti-PD1 or anti PDL1 antagonistic mAb.
said immunotherapeutic agent protein is etanercept.

According to another aspect the invention relates to a pharmaceutical composition for use in the treatment of T Cell Acute Lymphoblastic Leukemia (T-ALL), comprising a therapeutically effective amount of the anti-CD3 agent according to the invention and at least one immunotherapeutic agent according to the invention, and a pharmaceutically acceptable excipient.

According to other optional features of the pharmaceutical composition for use in the treatment of T Cell Acute Lymphoblastic Leukemia (T-ALL), it can optionally include one or more of the following characteristics alone or in combination:
said immunotherapeutic agent is selected among an anti-4-1BB agonistic mAb, an anti-OX40 agonistic mAb, an anti-PD1 antagonistic mAb, an anti-PDL1 antagonistic mAb and an TNFα/LTα antagonistic mAb, or a combination thereof;
said immunotherapeutic agent is a combination of at least two immunotherapeutic agents selected from an anti-4-1BB agonistic mAb, an anti-PD1 antagonistic mAb, an anti-PDL1 antagonistic mAb, an anti-OX40 agonistic mAb, and an anti-TNFα/LTα antagonistic mAb;
a therapeutically effective amount of an anti-CD3 agent as specified above in combination with at least one anti-4-1BB agonistic mAb and at least one anti-PD1 antagonistic mAb;
a therapeutically effective amount of an anti-CD3 agent as specified above in combination with at least one anti-4-1BB agonistic mAb and at least one anti-OX40 agonistic mAb;
a therapeutically effective amount of an anti-CD3 agent according as specified above in combination with at least one anti-4-1BB agonistic mAb, at least one anti-OX40 agonistic mAb and at least one anti-PD1 or anti PDL1 antagonistic mAb;
a therapeutically effective amount of an anti-CD3 agent as specified above in combination with an anti-4-1BB agonistic mAb, an anti-OX40 agonistic mAb and an anti-PD1 antagonistic mAb;
said immunotherapeutic agent is a Fc-fusion protein, in particular etanercept.

According to another aspect of the invention, it relates to an anti-CD3 agent for use in combination with at least one immunotherapeutic agent in a method for inducing cell death of T Cell Acute Lymphoblastic Leukemia (T-ALL) cells.

### DETAILED DESCRIPTION OF THE INVENTION

To understand the molecular basis of anti-CD3 mAbs anti-leukemic properties, the inventors performed microarray expression analysis and validation by Nanostring array and flow cytometry analysis of T-ALL PDX treated with a combination of the invention comprising anti-CD3 monoclonal antibodies with specific immunotherapeutic agents. Surprisingly, in addition to deregulated expression of apoptotic genes linked to thymocytes cell death, the inventors found upregulation of several pro-survival genes including PDCD1, encoding PD1 and its ligand PDL1 and a number of receptors (TNFR 2/TNFRSF1B, 4-1BB/TNFRSF9, OX 40/TNFRSF4, Fas/TNFRSF6, DR3/TNFRSF25, GITR/TNFRSF18), ligands (TNFα, LTα, TRAIL/TNFSF10, CD30L/TNFSF8, LIGHT/TNFSF14) and effectors (TRAF1, 3, 4, BIRC3) of the TNFR Super Family signaling pathways (FIG. 1A, B, C, D), while TNFR1/TNFRSF1A is constitutively expressed at the cell surface in T-ALL cells. The inventors next investigated the combination of an anti-CD3 agent with immunotherapeutic agents for treating cancer, in particular T-ALL. NSG mice carrying TCR+T-ALL patient-derived xenograft were co-treated with muromonab-CD3 mAb (also called anti-CD38 OKT3) and either anti-PD1 antagonistic mAbs (pembrolizumab or nivolumab), or with an anti-4-1BB agonistic mAb (urelumab) or with respective control IgG2a/IgG4 monoclonal antibodies, or with the TNFα/LTα inhibitor etanercept (ENBREL). Surprisingly, the inventors found that combination of an anti-CD3 agent with at least one immunotherapeutic agent allows a synergistic anti-leukemic effect between the two agents. Furthermore, such a combination enables induction of high caspase 8 activity in T-ALL cells and cancer cell death (FIG. 2).

Accordingly, the invention relates to an anti-CD3 agent for use in combination with at least one immunotherapeutic agent in the treatment of T Cell Acute Lymphoblastic Leukemia (T-ALL) in a subject in need thereof.

### Therapeutic Method

Accordingly, in a first aspect, the invention relates to an anti-CD3 agent for use in combination with at least one immunotherapeutic agent in the treatment of T Cell Acute Lymphoblastic Leukemia (T-ALL) in a subject in need thereof.

For the purpose of the invention, the terms “subject”, “individual” or “patient” are interchangeable and refer to a mammal, even more preferably to a human, including adult and child. Typically, a subject according to the invention refers to any subject, preferably human. In a particular embodiment, the term “subject” refers to a subject afflicted or at risk to be afflicted with cancer. In a particular embodiment, the term “subject” refers to a subject afflicted or at risk to be afflicted with Acute Lymphoblastic Leukemia (ALL). In a particular embodiment, the term “subject” refers to a subject afflicted or at risk to be afflicted with T Cell Acute Lymphoblastic Leukemia (T-ALL).

As used herein, the term “treatment” or “treat” refers to both prophylactic or preventive treatment as well as curative or disease modifying treatment, including treatment of subjects at risk of contracting the disease or disorder or suspected to have contracted the disease or disorder, as well as subjects who are ill or have been diagnosed as suffering from a disease or disorder, and includes suppression of clinical relapse. The treatment may be administered to a subject having a disease or disorder or who ultimately may acquire the disease or disorder, to: prevent, cure, delay the onset of, reduce the severity of, improve one or more symptoms of, delay or slow the progression of, prevent further progression of, in order to improve a subject's conditions and/or prolong its survival beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

The terms “combination”, “combination treatment” or “combination therapy” are understood to mean a combination whose base is made of an anti-CD3 agent and at least one immunotherapeutic agent co-administered to the subject for the purpose of achieving a biological effect. An anti-CD3 agent and at least one immunotherapeutic agent in this combination can be administered together or separately, concomitantly or sequentially. When administered together, they can be administered in a single composition comprising an anti-CD3 agent and at least one immunotherapeutic agent. In other words, said an anti-CD3 agent and at least one immunotherapeutic agent are thus formulated together. Alternatively, they can be administered separately to the subject, by the same or a different route of administration. Thus, for example, said at least one immunotherapeutic agent can be administered orally and said an anti-CD3 agent and at least one other compound can be injected into the subject, for example, intravenously or subcutaneously. In another embodiment, for example, said an anti-CD3 agent and said at least one immunotherapeutic agent are administered orally to the subject, but separately. Preferably, the sequence of administration of the active ingredients of the combination is such that said active ingredients or the active metabolites exert their biological effects at the same time, such that the subject benefits from the maximum effect of said combination. Thus, in a particularly preferred manner, an anti-CD3 agent and at least one immunotherapeutic agent are administered so as to reach their maximum concentration in the general blood circulation at the same time.

As used herein, an “agent” when referred to the treatment of T-ALL according to the invention, refers indifferently to the anti-CD3 agent or the immunotherapeutic agent individually, or even to the combination of said agents.

As used herein, except when otherwise specified, the article “a” or “an” carries the meaning of “one or more” or of “at least one”.

As used herein, the term “T Cell Acute Lymphoblastic Leukemia (T-ALL)” refers to a malignancy accounting for about 15% and 25% of ALL in pediatric and adult cohorts respectively. T-ALLs are a heterogeneous group of diseases regarding immunophenotype, cytogenetics, molecular genetic abnormalities and clinical features, including response to therapy (Reviewed in Belver & Ferrando, 2016; Girardi et al., 2017, Cordo et al., 2020). Cytogenetics, molecular approaches and mouse modeling have identified driver mutations in T-ALL (for reviews, see Belver and Ferrando 2016; Girardi et al., 2017; Cordo et al., 2020). Gene expression profiling is used to molecularly classify T-ALL in so-called immature/ETP-like, TLX, TLX1 /NKX2.1 and TAL/LMO subgroups. Early T cell progenitors (ETP) T-ALL includes the most immature cases, showing an expression profile shared with hematopoietic stem cells and early myeloid progenitors; the TLX group includes immature cases lacking a functional TCR al but often express cell surface TCR^{γδ} receptor. TLX1 /NKX2.1 T-ALL share genomic rearrangement in either TLX1 or NKX2.1 and a differentiation arrest at the cortical stage of T cell development. TAL/LMO T-ALL represent about half pediatric T-ALL cases, dysregulation of TAL/LMO expression as the result of various genetic alterations, a differentiation arrest close to mature thymocytes and TCR^{αβ} of expression. This molecular classification largely overlaps with differentiation arrest at different stages of T cell development as analyzed by immunophenotypic markers (Belver and Ferrando 2016).

Several immunophenotypic classifications have been proposed. The phenotypic classification proposed by the European Group for the Immunological Characterization of Leukemias (EGIL) is commonly used in Europe. According to EGIL, the presence of cytoplasmic or membrane expression of CD3 classifies T-ALL into four subgroups: (TI) the immature subgroup or pro-T-ALL is defined by the expression of only CD7; (TI1) pre-T-ALL express in addition CD2 and/or CD5 and/or CD8; (TI11) cortical T-ALL shows CDI a positivity; (TIV) mature T-ALL is characterized by the presence of surface CD3 and CD1 a negativity.

As used herein, the term “Cluster of Differentiation 3” or “CD3” has its general meaning in the art and refers to the protein complex associated with the T cell receptor, and which is essential to its intracellular signaling activities. It is composed of four distinct chains. In mammals, the complex contains a CD3-gamma (γ) chain, a CD3-delta (δ) chain and two CD3-epsilon (ε) chains. These chains associate with the TCR and the zeta-chain (ζ) to generate an activation signal in T lymphocytes. The TCR, zeta-chain (ζ) and CD3 molecules together constitute the TCR complex.

As used herein, the term “T Cell Receptor (TCR)” refers to a molecule found on the surface of T lymphocytes (or T cells) that is responsible for recognizing antigens bound to major histocompatibility complex (MHC) molecules. The TCR is composed of two different protein chains (i.e., heterodimer). In humans 95% of T cells TCR consists of an alpha (α) and beta (β) chain while 5% express a TCR composed of a delta (δ) and a gamma chain (γ).

As used herein, the term “anti-CD3 agent” has its general meaning in the art and refers to any agent selected from the group consisting of, but not limited to, compounds targeting the protein complex CD3. Accordingly, the term “anti-CD3 agent” refers to compounds that bind to CD3, CD3-gamma (γ) chain, CD3-delta (δ) chain, CD3-epsilon (ε) chains, or TCR complex and function as potent antagonists of CD3. The term “anti-CD3 agent” can also refer to a compound that selectively inactivates CD3, TCR complex, or CD3 intracellular signaling activities. Typically, an anti-CD3 agent is a small organic molecule, a protein, a polypeptide, a peptide, an oligopeptide, an aptamer, an oligonucleotide (antisense oligonucleotides, siRNA, shRNA, DNA and RNA aptamers), or an antibody. Anti-CD3 agents are well-known in the art as such as described in Kuhn et al., 2016, Alegre et al., 1992 and Menon et al., 2023.

The term “anti-CD3 agent” refers to any compound selected from but not limited to anti-CD3 monoclonal antibodies including but not limited to: foralumab (28F11-AE, NI-0401), teplizumab (hOKT3Y1, MGA031), muromonab-CD3 (anti-CD3 OKT3, OKT3, Orthoclone OKT3™), otelixizumab (TRX4, ChAglyCD3, GSK2136525), visilizumab (NUVION™, HuM291), blinatumomab (BLINCYTO™), catumaxomab (REMOVAB™), gOKT3-5 described in Kuhn et al., 2016 and Alegre et al., 1992.

Tests and assays for determining whether a compound is an anti-CD3 agent are well known by the skilled person in the art such as described in Kuhn et al., 2016, Alegre et al., 1992 and Menon et al., 2023. Determining whether a compound is an anti-CD3 agent may also be performed by using recombinant CD3 proteins, competitive binding assays and measuring the binding affinities, measuring the association of CD3 protein with the T cell receptor, measuring CD3 intracellular signaling activities, measuring the activation signal in T lymphocytes or assays such as described in the examples.

In an embodiment, the anti-CD3 agent for use according to the invention is an antibody, i.e., an anti-CD3 antibody.

As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods of the art to reduce its immunogenicity in man.

Antibodies are prepared according to conventional methodology. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of the target. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, the antigen may be provided as synthetic peptides corresponding to antigenic regions of interest in the target. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated a Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

This invention provides in certain embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria, which may be used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three-dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′) 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4. In a particular embodiment, the agent of the invention is a Human IgG4.

In another embodiment, the antibody according to the invention is a single domain antibody. The term “single domain antibody” (sdAb) or “VHH” refers to the single heavy chain variable domain of antibodies of the type that can be found in camelid mammals, which are naturally devoid of light chains. Such VHH are also called “nanobody®”. According to the invention, sdAb can particularly be llama sdAb. The term “VHH” refers to the single heavy chain having 3 complementarity determining regions (CDRs): CDR1, CDR2 and CDR3. The term “complementarity determining region” or “CDR” refers to the hypervariable amino acid sequences which define the binding affinity and specificity of the VHH.

The VHH according to the invention can readily be prepared by an ordinarily skilled artisan using routine experimentation. The VHH variants and modified form thereof may be produced under any known technique in the art such as in vitro maturation.

VHHs or sdAbs are usually generated by PCR cloning of the V-domain repertoire from blood, lymph node, or spleen cDNA obtained from immunized animals into a phage display vector, such as pHEN2. Antigen-specific VHHs are commonly selected by panning phage libraries on immobilized antigen, e.g., antigen coated onto the plastic surface of a test tube, biotinylated antigens immobilized on streptavidin beads, or membrane proteins expressed on the surface of cells. However, such VHHs often show lower affinities for their antigen than VHHs derived from animals that have received several immunizations. The high affinity of VHHs from immune libraries is attributed to the natural selection of variant VHHs during clonal expansion of B-cells in the lymphoid organs of immunized animals. The affinity of VHHs from non-immune libraries can often be improved by mimicking this strategy in vitro, i.e., by site directed mutagenesis of the CDR regions and further rounds of panning on immobilized antigen under conditions of increased stringency (higher temperature, high or low salt concentration, high or low pH, and low antigen concentrations). VHHs derived from camelid are readily expressed in and purified from the E. coli periplasm at much higher levels than the corresponding domains of conventional antibodies. VHHs generally display high solubility and stability and can also be readily produced in yeast, plant, and mammalian cells. For example, the “Hamers patents” describe methods and techniques for generating VHH against any desired target (see for example U.S. Pat. Nos. 5,800,988; 5,874, 541 and 6,015,695). The “Hamers patents” more particularly describe production of VHHs in bacterial hosts such as E. coli (see for example U.S. Pat. No. 6,765,087) and in lower eukaryotic hosts such as moulds (for example Aspergillus or Trichoderma) or in yeast (for example Saccharomyces, Kluyveromyces, Hansenula or Pichia) (see for example U.S. Pat. No. 6,838,254).

Although the anti-CD3 monoclonal antibodies as listed herein effective for their use according to the invention, affinity variants of anti-CD3 agents can be obtained starting from these antibodies. Affinity variants useful for the invention can exhibit an improved antagonistic activity toward CD3. In a particular example, these affinity variants can be antigen-binding fragments of the above listed anti-CD3 antibodies as described above (an antibody light chain variable domain (VL), an antibody heavy chain variable domain (VH), a single chain antibody (scFv), a F(ab′)2 fragment, a Fab fragment, an Fd fragment, an Fv fragment, and a single domain antibody fragment (DAb)), or results from recombinant means to combine features of the above described anti-CD3antibodies. Accordingly, in an embodiment, the anti-CD3 agent for use in a combination according to the invention is an affinity variant which competes for binding to CD3 with an antibody selected from the group consisting of foralumab, teplizumab, muromonab-CD3, otelixizumab, visilizumab, blinatumomab, catumaxomab (REMOVAB™), and/or gOKT3-5 as described above. A candidate competing antibody is considered an antibody that binds substantially to the same epitope or that competes for binding to the same epitope as the anti-CD3 antibody described herein, if the candidate competing antibody can block binding of the anti-CD3 antibody by at least 20%, preferably by at least 20-50%, even more preferably, by at least 50% as compared to a control, as using techniques well known in the art.

As used herein, the term “binding” in the context of the binding of an antibody to a predetermined antigen or epitope typically is a binding with an affinity corresponding to a KD of about 10-7 M or less, such as about 10-8 M or less, such as about 10-9 M or less, about 10-10 M or less, or about 10-11 M or even less when determined by for instance surface plasmon resonance (SPR) technology in a BIAcore 3000 instrument using a soluble form of the antigen as the ligand and the antibody as the analyte. BIACORE® (GE Healthcare, Piscaataway, NJ) is one of a variety of surface plasmon resonance assay format that are routinely used to epitope bin panels of monoclonal antibodies. Typically, an antibody binds to the predetermined antigen with an affinity corresponding to a KD that is at least ten-fold lower, such as at least 100-fold lower, for instance at least 1,000-fold lower, such as at least 10,000-fold lower, for instance at least 100,000-fold lower than its KD for binding to a non-specific antigen (e.g., BSA, casein), which is not identical or closely related to the predetermined antigen. When the KD of the antibody is very low (that is, the antibody has a high affinity), then the KD with which it binds the antigen is typically at least 10,000-fold lower than its KD for a non-specific antigen. An antibody is said to essentially not bind an antigen or epitope if such binding is either not detectable (using, for example, plasmon resonance (SPR) technology in a BIAcore 3000 instrument using a soluble form of the antigen as the ligand and the antibody as the analyte), or is 100 fold, 500 fold, 1000 fold or more than 1000 fold less than the binding detected by that antibody and an antigen or epitope having a different chemical structure or amino acid sequence.

Additional antibodies can be identified based on their ability to cross-compete (e.g., to competitively inhibit the binding of, in a statistically significant manner) with other antibodies of the invention in standard antigen binding assays. The ability of a test antibody to inhibit the binding of antibodies of the present invention to the target demonstrates that the test antibody can compete with that antibody for binding to the target; such an antibody may, according to non-limiting theory, bind to the same or a related (e.g., a structurally similar or spatially proximal) epitope on the target as the antibody with which it competes. Thus, another aspect of the invention provides antibodies that bind to the same antigen as, and compete with, the antibodies disclosed herein. As used herein, an antibody “competes” for binding when the competing antibody inhibits the target binding of an antibody or antigen binding fragment of the invention by more than 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% in the presence of an equimolar concentration of competing antibody.

In other embodiments the antibodies or antigen binding fragments of the invention bind to one or more epitopes of the target. In some embodiments, the epitopes to which the present antibodies or antigen binding fragments bind are linear epitopes. In other embodiments, the epitopes to which the present antibodies or antigen binding fragments bind are non-linear, conformational epitopes.

Antibodies directed against an antigen of interest (such as CD3) can be raised according to known methods by administering the appropriate antigen or epitope (e.g., a full-length CD3 protein or antigenic peptide fragment of CD3 in the case of CD3) to a host animal selected, e.g., from pigs, cows, horses, rabbits, goats, sheep, camelids (camel, dromedary, llama, vicuna) and mice, among others. Various adjuvants known in the art can be used to enhance antibody production.

In a specific embodiment, the anti-CD3 agent for use according to the invention is a monoclonal antibody (mAb).

As used herein, the term “monoclonal antibody” refers to an immunoglobulin (https://www.genscript.com/antibody-basics.html), generated from a single B-cell clone and which recognizes unique epitopes on a single antigen (such as CD3). Monoclonal antibodies can be prepared and isolated using any technique that provides for the production of antibody molecules by continuous cell lines in culture. Techniques for production and isolation include but are not limited to the hybridoma technique, the human B-cell hybridoma technique and the EBV-hybridoma technique using the method of Köhler (Köhler and Milstein 1975).

Several anti-CD3 monoclonal antibodies are known in the art, including, but not limited to: foralumab (28F11-AE, NI-0401), teplizumab (hOKT3Y1, MGA031), muromonab-CD3 (anti-CD3 OKT3, OKT3, Orthoclone OKT3™), otelixizumab (TRX4, ChAglyCD3, GSK2136525), visilizumab (NUVION™, HuM291), blinatumomab (BLINCYTO™), catumaxomab (REMOVAB™), and gOKT3-5 described in Kuhn et al., 2016 and Alegre et al., 1992.

In an embodiment, the anti-CD3 agent for use according to the invention is a monoclonal antibody selected from the group comprising: foralumab, teplizumab, otelixizumab, visilizumab, blinatumomab, and catumaxomab, or any combination thereof.

As seen previously, antibodies, and particularly monoclonal antibodies, can be mono or bispecific.

Thus, the anti-CD3 agent for use according to the invention can be a monospecific monoclonal antibody, selected from the group comprising: foralumab, teplizumab, otelixizumab and visilizumab, or any combination thereof.

In another embodiment, the anti-CD3 agent for use according to the invention can be a bispecific monoclonal antibody, selected from the group comprising: blinatumomab and catumaxomab, or any combination thereof.

In the present invention, the inventors show that combination between an anti-CD3 agent with at least one immunotherapeutic agent directly targets T-ALL cells and improves the therapeutic efficacy of said anti-CD3 agent in T-ALL in an unexpectedly synergistic manner.

In some embodiments, the invention relates to an anti-CD3 agent for use in combination with at least one, two, three, four, five, six, seven, eight, nine, ten, eleven, twelve or thirteen immunotherapeutic agents. As shown in the experiments combining, within the combinatory treatment according to the invention, several immunotherapeutic agents is of particular interest.

As used herein, the term “immunotherapeutic agent” is also used for “immunomodulating agent” and refers to an agent able to treat Acute Lymphoblastic Leukemia, preferably T Cell Acute Lymphoblastic Leukemia (T-ALL) either by directly killing leukemic cell and/or modulating the immune system of the subject afflicted by the T-ALL, by stimulating or suppressing the patient's immune system to attack the malignant tumor cells.

The immunotherapeutic agent according to the invention can be a small organic molecule, a protein, a fusion protein, a polypeptide, a peptide, an oligopeptide, an aptamer (Soldevilla et al., 2016), an oligonucleotide (such as antisense oligonucleotides, siRNA, shRNA, DNA and RNA aptamers), or an antibody.

As used herein, the term “immunotherapeutic agent” also refers to a cancer therapeutic treatment using the immune system to reject cancer. The therapeutic treatment stimulates the patient's immune system to attack the malignant tumor cells.

The term “immunotherapeutic agent” also refers to immune checkpoint therapy such as checkpoint inhibitors including, but are not limited to programmed death-1 (PD-1) inhibitors, programmed death ligand-1 (PD-L1) inhibitors, programmed death ligand-2 (PD-L2) inhibitors, lymphocyte-activation gene 3 (LAG3) inhibitors, T-cell immunoglobulin and mucin-domain containing protein 3 (TIM-3) inhibitors, T cell immunoreceptor with Ig and ITIM domains (TIGIT) inhibitors, B-and T-lymphocyte attenuator (BTLA) inhibitors, V-domain Ig suppressor of T-cell activation (VISTA) inhibitors, cytotoxic T-lymphocyte-associated protein 4 (CTLA4) inhibitors, Indoleamine 2,3-dioxygenase (IDO) inhibitors, killer immunoglobulin-like receptors (KIR) inhibitors, KIR2L3 inhibitors, KIR3DL2 inhibitors and carcinoembryonic antigen-related cell adhesion molecule 1 (CEACAM-1) inhibitors. In particular, checkpoint inhibitors include antibodies anti-PD1, anti-PD-L1, anti-CTLA4, anti-TIM-3, anti-LAG3. Immune checkpoint therapy also includes co-stimulatory antibodies delivering positive signals through immune-regulatory receptors including but not limited to ICOS, CD137, CD27, OX40 and GITR.

Cell surface glycoprotein 4-1BB (also known as CD 137 and TNFRSF 9) is a member of the tumor necrosis factor receptor super family (TNFRSF) that is expressed on activated T cells, among others. Activation of 4-1BB by its 4-1BB ligand delivers potent costimulatory signals to CD8+cytotoxic T cells, promoting cell proliferation, facilitating differentiation into memory cells, and delivering important survival signal. However, under certain circumstances, 4-1BB signaling can deliver an apoptotic signal to activated T-cells (Ma BY et al., 2005).

Preclinical studies have been performed using 4-1BB agonistic monoclonal antibodies (i.e., anti-4-1BB agonistic mAb), such as urelumab (BMS-663513) or utomilumab (PF-05082566). For example, Eskiocak et al., showed that anti-4-1BB agonistic mAb are pre-clinically efficient at blocking growth of solid tumors through their ability to co-activate anti-tumor T cells cytotoxic activity and NK cells ADCC (Eskiocak et al., 2020). Novel therapeutic strategies aimed to minimize cytotoxicity of available agonistic mAbs are under development (Chester C. et al., 2018).

OX40 (also known as TNFRSF4 or CD134) is another member of the TNFR super family (TNFRSF) and is type I transmembrane glycoproteins characterized by the presence of cysteine-rich domains (CRDs). The only ligand known for OX40 is OX40L. OX40 L/OX40 interaction delivers co-stimulatory signals to antigens-stimulated T cells but can also deliver an apoptotic signal (Ma BY et al., 2005) Clinical trials are in progress on anti-OX40 agonistic monoclonal antibodies (i.e., anti-OX40 agonistic mAb), such as MEDI6469 (9B12), MEDI0562, PF-04518600, INCAGN01949, BMS-986178, MOXR0916 (RG7888), GSK3174998 or MEDI6383. Notably, several clinical trials are ongoing using an anti-OX40 agonistic mAb in association with other therapies in solid tumors (Buchan et al., 2018).

Programmed cell death 1 PD-1 (also called CD279) is an immune checkpoint receptor playing a critical role in immunity. Briefly, it regulates the threshold of antigen responses of T cells and B cells. Its ligands are PDL1 (B7-H1 or CD274) and PD-L2 (B7-DC or CD273) and both PD-1 and PDL1 are type I transmembrane proteins that belong to the immunoglobulin (Ig) superfamily.

Several antagonistic monoclonal antibodies have been developed against PD-1 (i.e., anti-PD1 antagonistic mAb), such as cemiplimab, dostarlimab, nivolumab, pembrolizumab or pidilizumab (CT-011). Antagonistic anti-PD1 or anti-PDL1 (atezolizumab, durvalumab, avelumab) therapeutic antibodies are widely used clinically to relieve the Immune T cells exhaustion program in patients and restore an active anti-tumor response (Pardoll et al., 2012).

There are two receptors for the related tumor necrosis alpha (TNFα encoded by the TNFA gene) and lymphotoxin alpha (LTα encoded by LTA gene): the ubiquitously expressed TNF receptor 1 (TNFR1) and 2 (TNFR2), mostly confined to immune cells. Binding of TNFα/LTα to TNFR1 induces the recruitment to the cytoplasmic domain of TNFR1 of a TRADD/TRAF2 /RIPK1/c-IAP1/2 protein complex (complex 1) that leads to activation of NF-kB and MAP Kinase pathways favoring cell survival/proliferation. Upon internalization or e.g., c-IAP1/2 loss-of-function, cytoplasmic complexes are formed (complex 2a and b) including FAS-associated death domain (FADD), RIPK1, RIPK3 and pro-caspase 8, that leads to cell death. Binding of TNFα/LTα to TNFR2 recruits TRAF2 /c-IAP1/c-IAP2 complexes, activation of both canonical and non-canonical NF-kB, MAP Kinase and AKT, associated with cell survival/proliferation. TNFR2 can also induce cell death either directly or indirectly by crosstalk with TNFR1. Several inhibitors of TNFα/LTα have been and continue to be developed including monoclonal antibodies (infliximab, adalimumab, certolizumab, golimumab), the TNFR2-Fc fusion protein etanercept (ENBREL™) or low-molecular weight small chemicals (reviewed in Jang et al., 2021; Javaid N. et al., 2022).

Example of anti-4-1BB antibodies include, but are not limited to, urelumab or utomilumab. Example of anti-OX40 antibodies include, but are not limited to, MEDI6469 (9B12), MEDI0562, PF-04518600, INCAGN01949, BMS-986178, MOXR0916 (RG7888), GSK3174998 or MEDI6383. Example of anti-PD1 antibodies include, but are not limited to, cemiplimab (REGN2810 or REGN-2810, LIBTAYO™), tislelizumab (BGB-A317), tislelizumab, spartalizumab (PDR001 or PDR-001), ABBV-181, JNJ-63723283, BI 754091, MAG012, TSR-042, AGEN2034, nivolumab (ONO-4538, BMS-936558, MDX1106, GTPL7335 or OPDIVO™), pembrolizumab (MK-3475, MK03475, lambrolizumab, SCH-900475 or KEYTRUDA™), dostarlimab (JEMPERLI™), pidilizumab (CT-011) and antibodies described in International patent applications WO2004004771, WO2004056875, WO2006121168, WO2008156712, WO2009014708, WO2009114335, WO2013043569 and WO2014047350. Example of anti-PD-L1 antibodies include, but are not limited to, LY3300054, Atezolizumab, Durvalumab and Avelumab. Example of TNFα/LTα antibodies include, but are not limited to, infliximab, adalimumab, certolizumab, golimumab. Example of anti-CTLA4 antibodies include, but are not limited to, Ipilimumab (see, e.g., U.S. Pat. Nos. 6,984,720 and 8,017,114), tremelimumab (see, e.g., U.S. Pat. Nos. 7,109,003 and 8,143,379), single chain anti-CTLA4 antibodies (see, e.g., International patent applications WO1997020574 and WO2007123737) and antibodies described in U.S. Pat. No. 8,491,895. Example of anti-VISTA antibodies are described in US patent application US20130177557. Example of inhibitors of the LAG3 receptor are described in U.S. Pat. No. 5,773,578. Example of KIR inhibitor is IPH4102 targeting KIR3DL2.

In an embodiment, the at least one immunotherapeutic agent to be combined with an anti-CD3 for use according to the invention is a monoclonal antibody targeting at least one target selected from the group consisting of: 4-1BB, OX40, PD-1, PDL 1, TRAIL, TNFR1, TNFR2, TNFα/LTα, CD30, CD38, and/or CTLA4.

In some embodiment, the immunotherapeutic agent is an anti-TRAIL agent such as described in Chodorge et al., 2012, an anti-TNFR 1 agent, an anti-TNFR2 agent, an anti-TNFα/LTα agent such as infliximab, adalimumab, certolizumab, or golimumab, an anti-CD30 agent such as brentuximab, an anti-CD38 agent such as daratumumab, an anti-FAS agent or an anti-GITR agent.

In some embodiment, the immunotherapeutic agent is a monoclonal antibody targeting at least one target selected from the group consisting of: 4-1BB, OX40, PD-1 and/or TNFα/LTα.

In a particular embodiment, the immunotherapeutic agent is a monoclonal antibody targeting at least one target selected from the group consisting of: 4-1BB, OX40 and/or PD-1.

In one embodiment, the immunotherapeutic agent is selected among anti-4-1BB mAb, anti-OX40 mAb, anti-PD1 mAb, anti-PDL1 and anti-TNFα/LTα mAb, or any combination thereof.

In one embodiment, the immunotherapeutic agent is selected among anti-4-1BB agonistic mAb, anti-OX40 agonistic mAb, anti-PD1 antagonistic mAb, anti-PDL1 antagonistic mAb and/or anti-TNFα/LTα antagonist.

In some embodiment, the immunotherapeutic agent is selected from the group consisting of: urelumab, utomilumab, MEDI6469 (9B12), MEDI0562, PF-04518600, INCAGN01949, BMS-986178, MOXR0916 (RG7888), GSK3174998, MEDI6383, cemiplimab, dostarlimab, nivolumab, pembrolizumab, pidilizumab (CT-011), atezolizumab, durvalumab, avelumab, infliximab, adalimumab, certolizumab and golimumab, or any combination thereof.

In a particular embodiment, the immunotherapeutic agent is selected from the group consisting of: urelumab, nivolumab, and pembrolizumab, or any combination thereof.

More particularly, the immunotherapeutic agent is selected from the group consisting of:
urelumab,
nivolumab,
pembrolizumab,
urelumab and nivolumab,
nivolumab and pembrolizumab, and
urelumab and pembrolizumab.

In some embodiment, the at least one immunotherapeutic agent to be combined with an anti-CD3 for use according to the invention is a fusion protein.

A fusion protein consists of two proteins that have been joined into single unit. This type of protein typically retains some characteristics of both original proteins. Fusion proteins can occur naturally but are often engineered to help purify a protein, or to aid in the study of its expression or localization.

Fc-fusion proteins are specific type of fusion proteins and are composed of Fc region of IgG antibody (Hinge-CH2-CH3) and a desired linked protein. Fc region of Fc-fusion proteins can bind to neonatal Fc receptor (FcRn) thereby rescuing it from degradation. Fc-fusion proteins can potentially be useful in some therapeutic approaches, such as treatment of HIV, inflammatory diseases, diabetes, or cancer. The linked protein may be an interested protein such as receptor, cytokine, enzyme, or peptide (peptibody).

In some embodiment, the immunotherapeutic agent is a Fc-fusion protein.

In an embodiment, the immunotherapeutic agent is a Fc-fusion protein selected among: etanercept (TNFR2-Fc fusion protein or ENBREL™), alefacept, abatacept, rilonacept, romiplostim, belatacept, aflibercept, Eloctate, Alprolix, dulaglutide, trebananib, blisibimod, apocept, CNTO 528, CNTO 530, NKG2D-Fc or ALT-803.

In some embodiment, the at least one immunotherapeutic agent to be combined with an anti-CD3 for use according to the invention etanercept.

Several combinations of anti-CD3 agent, as previously described, with at least one immunotherapeutic agent, as previously described, can be used for the treatment of T-ALL.

In an embodiment, the anti-CD3 agent is used according to the invention in combination with at least one immunotherapeutic agent selected among anti-4-1BB agonistic mAb, anti-OX40 agonistic mAb, anti-PD1 antagonistic mAb, anti-PDL1 antagonistic mAb, anti-TRAIL agonistic mAb, anti-TNFR1 antagonistic mAb, anti-TNFR2 antagonistic mAb, anti-TNFα/LTα antagonistic mAb, or any combination thereof.

In a particular embodiment, the at least one immunotherapeutic agent is selected among anti-4-1BB agonistic mAb, anti-OX40 agonistic mAb, anti-PD1 antagonistic mAb, anti-PDL1 antagonistic mAb and anti-TNFα/LTα antagonistic mAb or any combination thereof.

In a particular embodiment, the anti-CD3 agent is used according to the invention in combination with an anti-4-1BB agonistic mAb and an anti-OX40 agonistic mAb.

In another particular embodiment, the anti-CD3 agent is used according to the invention in combination with an anti-4-1BB agonistic mAb and an anti-PD1 antagonistic mAb.

In further particular embodiment, the anti-CD3 agent is used according to the invention in combination with an anti-4-1BB agonistic mAb and an anti-PDL1 antagonistic mAb.

In further particular embodiment, the anti-CD3 agent is used according to the invention in combination with an anti-4-1BB agonistic mAb and an anti-TNFα/LTα antagonistic mAb.

In a specific embodiment, the anti-CD3 agent is used according to the invention in combination with an anti-4-1BB agonistic mAb, an anti-OX40 agonistic mAb and an anti-PD1 antagonistic mAb.

In another specific embodiment, the anti-CD3 agent is used according to the invention in combination with an anti-4-1BB agonistic mAb, an anti-OX40 agonistic mAb and an anti-PDL1 antagonistic mAb.

In an embodiment, the anti-CD3 agent is used according to the invention in combination with an anti-4-1BB agonistic mAb, an anti-OX40 agonistic mAb and aTNFa/LTα antagonist.

In an embodiment, the anti-CD3 agent for use according to the invention is selected from the group consisting of foralumab, teplizumab, otelixizumab, visilizumab, blinatumomab, and catumaxomab and the immunotherapeutic agent is selected from the group consisting of urelumab, utomilumab, MEDI6469, MEDI0562, PF-04518600, INCAGN01949, BMS-986178, MOXR0916, GSK3174998, MEDI6383, cemiplimab, dostarlimab, nivolumab, pembrolizumab, pidilizumab (CT-011), atezolizumab, durvalumab, avelumab, infliximab, adalimumab, certolizumab, golimumab and etanercept, or any combination thereof.

In a specific embodiment, the anti-CD3 agent for use according to the invention is selected from the group consisting of foralumab, teplizumab, otelixizumab and visilizumab and the immunotherapeutic agent is selected from the group consisting of urelumab, utomilumab, MEDI6469, MEDI0562, PF-04518600, INCAGN01949, BMS-986178, MOXR0916, GSK3174998, MEDI6383, cemiplimab, dostarlimab, nivolumab, pembrolizumab, pidilizumab (CT-011), atezolizumab, durvalumab, avelumab, infliximab, adalimumab, certolizumab, golimumab and etanercept, or any combination thereof.

In a more specific embodiment, the anti-CD3 agent for use according to the invention is selected from the group consisting of foralumab and teplizumab, or any combination thereof, and the immunotherapeutic agent is selected from the group consisting of foralumab, teplizumab, otelixizumab and/or visilizumab and the immunotherapeutic agent is selected from the group consisting of urelumab, utomilumab, MEDI6469, MEDI0562, PF-04518600, INCAGN01949, BMS-986178, MOXR0916, GSK3174998, MEDI6383, cemiplimab, dostarlimab, nivolumab, pembrolizumab, pidilizumab (CT-011), atezolizumab, durvalumab, avelumab, infliximab, adalimumab, certolizumab, golimumab and etanercept, or any combination thereof.

In an even more specific embodiment, the anti-CD3 agent for use according to the invention is foralumab and the immunotherapeutic agent is selected from the group consisting of urelumab, utomilumab, MEDI6469, MEDI0562, PF-04518600, INCAGN01949, BMS-986178, MOXR0916, GSK3174998, MEDI6383, cemiplimab, dostarlimab, nivolumab, pembrolizumab, pidilizumab (CT-011), atezolizumab, durvalumab, avelumab, infliximab, adalimumab, certolizumab, golimumab and etanercept, or any combination thereof.

In a further more specific embodiment, the anti-CD3 agent for use according to the invention is teplizumab and the immunotherapeutic agent is selected from the group consisting of urelumab, utomilumab, MEDI6469, MEDI0562, PF-04518600, INCAGN01949, BMS-986178, MOXR0916, GSK3174998, MEDI6383, cemiplimab, dostarlimab, nivolumab, pembrolizumab, pidilizumab (CT-011), atezolizumab, durvalumab, avelumab, infliximab, adalimumab, certolizumab, golimumab and etanercept, or any combination thereof.

In another specific embodiment, the anti-CD3 agent for use according to the invention is muromonab-CD3 and the immunotherapeutic agent is selected from the group consisting of urelumab, utomilumab, MEDI6469, MEDI0562, PF-04518600, INCAGN01949, BMS-986178, MOXR0916, GSK3174998, MEDI6383, cemiplimab, dostarlimab, nivolumab, pembrolizumab, pidilizumab (CT-011), atezolizumab, durvalumab, avelumab, infliximab, adalimumab, certolizumab, golimumab and etanercept, or any combination thereof.

In another specific embodiment, the anti-CD3 agent for use according to the invention is otelixizumab and the immunotherapeutic agent is selected from the group consisting of urelumab, utomilumab, MEDI6469, MEDI0562, PF-04518600, INCAGN01949, BMS-986178, MOXR0916, GSK3174998, MEDI6383, cemiplimab, dostarlimab, nivolumab, pembrolizumab, pidilizumab (CT-011), atezolizumab, durvalumab, avelumab, infliximab, adalimumab, certolizumab, golimumab and etanercept, or any combination thereof.

In another specific embodiment, the anti-CD3 agent for use according to the invention is visilizumab and the immunotherapeutic agent is selected from the group consisting of urelumab, utomilumab, MEDI6469, MEDI0562, PF-04518600, INCAGN01949, BMS-986178, MOXR0916, GSK3174998, MEDI6383, cemiplimab, dostarlimab, nivolumab, pembrolizumab, pidilizumab (CT-011), atezolizumab, durvalumab, avelumab, infliximab, adalimumab, certolizumab, golimumab and etanercept, or any combination thereof.

In another specific embodiment, the anti-CD3 agent for use according to the invention is blinatumomab and the immunotherapeutic agent is selected from the group consisting of urelumab, utomilumab, MEDI6469, MEDI0562, PF-04518600, INCAGN01949, BMS-986178, MOXR0916, GSK3174998, MEDI6383, cemiplimab, dostarlimab, nivolumab, pembrolizumab, pidilizumab (CT-011), atezolizumab, durvalumab, avelumab, infliximab, adalimumab, certolizumab, golimumab and etanercept, or any combination thereof.

In another specific embodiment, the anti-CD3 agent for use according to the invention is catumaxomab and the immunotherapeutic agent is selected from the group consisting of urelumab, utomilumab, MEDI6469, MEDI0562, PF-04518600, INCAGN01949, BMS-986178, MOXR0916, GSK3174998, MEDI6383, cemiplimab, dostarlimab, nivolumab, pembrolizumab, pidilizumab (CT-011), atezolizumab, durvalumab, avelumab, infliximab, adalimumab, certolizumab, golimumab and etanercept, or any combination thereof.

In a particular embodiment, the anti-CD3 agent for use according to the invention is teplizumab and the immunotherapeutic agent is selected from the group of combinations consisting of:
urelumab and nivolumab,
nivolumab and pembrolizumab, and
urelumab and pembrolizumab.

In a particular embodiment, the anti-CD3 agent for use according to the invention is foralumab and the immunotherapeutic agent is selected from the group of combinations consisting of:
urelumab and nivolumab,
nivolumab and pembrolizumab, and
urelumab and pembrolizumab.

In a more specific embodiment, the anti-CD3 agent for use according to the invention is selected from the group consisting of foralumab and teplizumab, or any combination thereof, and the immunotherapeutic agent is selected from the group consisting of urelumab, nivolumab, pembrolizumab, and etanercept or any combination thereof.

In an even more specific embodiment, the anti-CD3 agent for use according to the invention is foralumab and the immunotherapeutic agent is selected from the group consisting of urelumab, nivolumab, pembrolizumab, and etanercept or any combination thereof.

In another specific embodiment, the anti-CD3 agent for use according to the invention is teplizumab and the immunotherapeutic agent is selected from the group consisting of urelumab, nivolumab, pembrolizumab, and etanercept or any combination thereof.

In a particular embodiment, the anti-CD3 agent for use according to the invention is muromonab-CD3 and the immunotherapeutic agent is selected from the group of combinations consisting of:
urelumab and nivolumab,
nivolumab and pembrolizumab, and
urelumab and pembrolizumab.

In a particular embodiment, the anti-CD3 agent for use according to the invention is otelixizumab and the immunotherapeutic agent is selected from the group of combinations consisting of:
urelumab and nivolumab,
nivolumab and pembrolizumab, and
urelumab and pembrolizumab.

In a particular embodiment, the anti-CD3 agent for use according to the invention is visilizumab and the immunotherapeutic agent is selected from the group of combinations consisting of:
urelumab and nivolumab,
nivolumab and pembrolizumab, and
urelumab and pembrolizumab.

In a particular embodiment, the anti-CD3 agent for use according to the invention is blinatumomab and the immunotherapeutic agent is selected from the group of combinations consisting of:
urelumab and nivolumab,
nivolumab and pembrolizumab, and
urelumab and pembrolizumab.

In a particular embodiment, the anti-CD3 agent for use according to the invention is catumaxomab and the immunotherapeutic agent is selected from the group of combinations consisting of:
urelumab and nivolumab,
nivolumab and pembrolizumab, and
urelumab and pembrolizumab.

In another aspect, the invention relates to an anti-CD3 agent for use in combination with at least one immunotherapeutic agent in a method for inducing cell death of T Cell Acute Lymphoblastic Leukemia.

The anti-CD3 agent and immunotherapeutic agent are defined as previously described.

In a further aspect, the invention relates to a method for treating T Cell Acute Lymphoblastic Leukemia (T-ALL) in a subject in need thereof, comprising administering to said subject a therapeutically effective amount of an anti-CD3 agent in combination with at least one immunotherapeutic agent as described above.

In a specific embodiment, the invention relates to a method for treating T Cell Acute Lymphoblastic Leukemia (T-ALL) in a subject in need thereof, comprising administering to said subject a therapeutically effective amount of an anti-CD3 monoclonal antibody in combination with at least one monoclonal antibody and/or at least one Fc-fusion protein as immunotherapeutic agent.

In another aspect, the invention relates to a combination of an anti-CD3 agent as described above with a SMAC-mimetic for use in the treatment of T-ALL.

Members of the inhibitor of apoptosis protein (IAP) family are negative regulators of caspase activation and can be inhibited by the second mitochondrial-derived activator of caspases (SMAC). The inventors observed that combination of an anti-CD3 agent according to the invention with a SMAC mimetic, such as birinapant, induces a synergistic anti-leukemic effect.

SMAC is a mitochondrial protein that binds inhibitor of apoptosis proteins, inhibiting IAPs ability to bind caspases, which are pro-apoptotic proteins. IAPs antagonistically bind caspases, thus, SMAC induces a pro-apoptotic effect. Endogenous SMAC binds the Bir-domain of c-IAP1, cIAP2 and XIAP via an IAP-binding motif (IBM). Endogenous SMAC homodimerizes and is bivalent.

In an embodiment, the SMAC mimetic is an IAP antagonist, in particular an c-IAP1/2 inhibitor.

In a more particular embodiment, the invention relates to a synergistic combination of an anti-CD3 agent as described above with a SMAC-mimetic for use in the treatment of T-ALL. In an even more particular embodiment, the invention relates to a synergistic combination of an anti-CD3 agent as described above with an IAP antagonist, in particular an c-IAP1/2 inhibitor, for use in the treatment of T-ALL. It has indeed been observed that cIAP inhibition synergizes with anti-CD3 in T-ALL.

In an embodiment, the SMAC mimetic can be selected in the group consisting of: birinapant (CAS registry No 1260251-31-7), APG-1387 (CAS registry No 1570231-89-8), Debio 1143 (also called xevinapant, CAS registry No 1071992-99-8), ASTX 660 (CAS registry No 1799328-86-1), GDC-0152 (CAS registry No 873652-48-3), HGS-1029/AEG 40826 (CAS registry No 1107664-44-7), SM-164 (CAS registry No 957135-43-2), BV6 (CAS registry No 1001600-56-1), embelin 15 (CAS registry No 550-24-3), MV1 (CAS registry No 1001600-54-9), SM-1295 (CAS registry No 1562375-46-5), SBP-0636457 (CAS registry No 1422180-49-1), GDC-0917 (CAS registry No 1446182-94-0), LCL 161 (CAS registry No 1005342-46-0), XIAP/cIAP1 antagonist-1 (CAS registry No 1403898-55-4), BI 891065 (Boehringer Ingelheim) and AZD 5582 (CAS registry No 1258392-53-8). Preferably, the SMAC mimetic is birinapant.

In a specific embodiment, the anti-CD3 agent for use according to the invention selected from foralumab, teplizumab, otelixizumab, visilizumab, blinatumomab, and catumaxomab or any combination thereof, is combined with an IAP antagonist, in particular an c-IAP1/2 inhibitor.

In a more specific embodiment, the anti-CD3 agent for use according to the invention is foralumab and a SMAC mimetic is selected in the group consisting of: birinapant, APG-1387, debio 1143, ASTX660, GDC-0152, HGS-1029/AEG40826, SM-164, BV6, embelin 15, MV1, SM-1295, SBP-0636457, GDC-0917, LCL161, XIAP/cIAP1 antagonist-1, BI 891065 and AZD5582, or any combination thereof.

In a more specific embodiment, the anti-CD3 agent for use according to the invention is teplizumab and a SMAC mimetic is selected in the group consisting of: birinapant, APG-1387, debio 1143, ASTX660, GDC-0152, HGS-1029/AEG40826, SM-164, BV6, embelin 15, MV1, SM-1295, SBP-0636457, GDC-0917, LCL161, XIAP/cIAP1 antagonist-1, BI 891065 and AZD5582, or any combination thereof.

In a more specific embodiment, the anti-CD3 agent for use according to the invention is muromonab-CD3 and a SMAC mimetic is selected in the group consisting of: birinapant, APG-1387, debio 1143, ASTX660, GDC-0152, HGS-1029/AEG40826, SM-164, BV6, embelin 15, MV1, SM-1295, SBP-0636457, GDC-0917, LCL161, XIAP/cIAP1 antagonist-1, BI 891065 and AZD5582, or any combination thereof.

In a more specific embodiment, the anti-CD3 agent for use according to the invention is otelixizumab and SMAC mimetic is selected in the group consisting of: birinapant, APG-1387, debio 1143, ASTX660, GDC-0152, HGS-1029/AEG40826, SM-164, BV6, embelin 15, MV1, SM-1295, SBP-0636457, GDC-0917, LCL161, XIAP/cIAP1 antagonist-1, BI 891065 and AZD5582, or any combination thereof.

In a more specific embodiment, the anti-CD3 agent for use according to the invention is visilizumab and SMAC mimetic is selected in the group consisting of: birinapant, APG-1387, debio 1143, ASTX660, GDC-0152, HGS-1029/AEG40826, SM-164, BV6, embelin 15, MV1, SM-1295, SBP-0636457, GDC-0917, LCL161, XIAP/cIAP1 antagonist-1, BI 891065 and AZD5582, or any combination thereof.

In a more specific embodiment, the anti-CD3 agent for use according to the invention is blinatumomab and SMAC mimetic is selected in the group consisting of: birinapant, APG-1387, debio 1143, ASTX660, GDC-0152, HGS-1029/AEG40826, SM-164, BV6, embelin 15, MV1, SM-1295, SBP-0636457, GDC-0917, LCL161, XIAP/cIAP1 antagonist-1, BI 891065 and AZD5582, or any combination thereof.

In a more specific embodiment, the anti-CD3 agent for use according to the invention is catumaxomab and SMAC mimetic is selected in the group consisting of: birinapant, APG-1387, debio 1143, ASTX660, GDC-0152, HGS-1029/AEG40826, SM-164, BV6, embelin 15, MV1, SM-1295, SBP-0636457, GDC-0917, LCL161, XIAP/cIAP1 antagonist-1, BI 891065 and AZD5582, or any combination thereof.

In a particular embodiment, the invention relates to a combination of an anti-CD3 agent as described above with birinapant for use in the treatment of T-ALL.

In some embodiments, the compound, combination and/or pharmaceutical composition of the invention may be used in combination with targeted therapy. As used herein, the term “targeted therapy” refers to targeted therapy agents, drugs designed to interfere with specific molecules necessary for tumor growth and progression. For example, targeted therapy agents such as therapeutic monoclonal antibodies target specific antigens found on the cell surface, such as transmembrane receptors or extracellular growth factors. Small molecules can penetrate the cell membrane to interact with targets inside a cell. Small molecules are usually designed to interfere with the enzymatic activity of the target protein such as for example proteasome inhibitor, tyrosine kinase or cyclin-dependent kinase inhibitor, histone deacetylase inhibitor. Targeted therapy may also use cytokines. Examples of such targeted therapy include with no limitations: Ado-trastuzumab emtansine (HER2), afatinib (EGFR (HER1/ERBB1), HER2), aldesleukin (Proleukin), alectinib (ALK), alemtuzumab (CD52), axitinib (kit, PDGFRbeta, VEGFR1/2/3), belimumab (BAFF), belinostat (HDAC), bevacizumab (VEGF ligand), blinatumomab (CD19/CD3), bortezomib (proteasome), brentuximab vedotin (CD30), bosutinib (ABL), brigatinib (ALK), cabozantinib (FLT3, KIT, MET, RET, VEGFR2), canakinumab (IL-1 beta), carfilzomib (proteasome), ceritinib (ALK), cetuximab (EGFR), cofimetinib (MEK), crizotinib (ALK, MET, ROS1), dabrafenib (BRAF), daratumumab (CD38), dasatinib (ABL), denosumab (RANKL), dinutuximab (B4GALNT1 (GD2)), elotuzumab (SLAMF7), enasidenib (IDH2), erlotinib (EGFR), everolimus (mTOR), gefitinib (EGFR), ibritumomab tiuxetan (CD20), sonidegib (Smoothened), sipuleucel-T, siltuximab (IL-6), sorafenib (VEGFR, PDGFR, KIT, RAF), tocilizumab (IL-6R), temsirolimus (mTOR), tofacitinib (JAK3), trametinib (MEK), tositumomab (CD20), trastuzumab (HER2), vandetanib (EGFR), vemurafenib (BRAF), venetoclax (BCL2), vismodegib (PTCH, Smoothened), vorinostat (HDAC), ziv-aflibercept (PIGF, VEGFA/B), olaparib (PARP inhibitor).

In some embodiments, the compound, combination and/or pharmaceutical composition of the invention may be used in combination with chemotherapy. As used herein, the term “antitumor chemotherapy” or “chemotherapy” has its general meaning in the art and refers to a cancer therapeutic treatment using chemical or biochemical substances, in particular using one or several antineoplastic agents or chemotherapeutic agents. Chemotherapeutic agents include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; methylhydrazine derivatives including N-methylhydrazine (MIH) and procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-1 1); topoisomerase inhibitor RFS 2000; difluoromethylomithine (DMFO); retinoids such as retinoic acid; capecitabine; anthracyclines, nitrosoureas, antimetabolites, epipodophylotoxins, enzymes such as L-asparaginase; anthracenediones; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide; and pharmaceutically acceptable salts, acids or derivatives of any of the above.

In some embodiments, the compound, combination and/or pharmaceutical composition of the invention is administered to the subject in combination with radiotherapy. Suitable examples of radiation therapies include, but are not limited to external beam radiotherapy (such as superficial X-rays therapy, orthovoltage X-rays therapy, megavoltage X-rays therapy, radiosurgery, stereotactic radiation therapy, Fractionated stereotactic radiation therapy, cobalt therapy, electron therapy, fast neutron therapy, neutron-capture therapy, proton therapy, intensity modulated radiation therapy (IMRT), 3-dimensional conformal radiation therapy (3D-CRT) and the like); brachytherapy; unsealed source radiotherapy; tomotherapy; and the like. Gamma rays are another form of photons used in radiotherapy. Gamma rays are produced spontaneously as certain elements (such as radium, uranium, and cobalt 60) release radiation as they decompose, or decay. In some embodiments, radiotherapy may be proton radiotherapy or proton minibeam radiation therapy. Proton radiotherapy is an ultra-precise form of radiotherapy that uses proton beams (Prezado Y, Jouvion G, Guardiola C, Gonzalez W, Juchaux M, Bergs J, Nauraye C, Labiod D, De Marzi L, Pouzoulet F, Patriarca A, Dendale R. Tumor Control in RG2 Glioma-Bearing Rats: A Comparison Between Proton Minibeam Therapy and Standard Proton Therapy. Int J Radiat Oncol Biol Phys. 2019 Jun. 1; 104(2):266-271. doi: 10.1016/j.ijrobp.2019.01.080; Prezado Y, Jouvion G, Patriarca A, Nauraye C, Guardiola C, Juchaux M, Lamirault C, Labiod D, Jourdain L, Sebrie C, Dendale R, Gonzalez W, Pouzoulet F. Proton minibeam radiation therapy widens the therapeutic index for high-grade gliomas. Sci Rep. 2018 Nov. 7; 8(1):16479. doi: 10.1038/s 41598-018-34796-8). Radiotherapy may also be FLASH radiotherapy (FLASH-RT) or FLASH proton irradiation. FLASH radiotherapy involves the ultra-fast delivery of radiation treatment at dose rates several orders of magnitude greater than those currently in routine clinical practice (ultra-high dose rate) (Favaudon V, Fouillade C, Vozenin MC. The radiotherapy FLASH to save healthy tissues. Med Sci (Paris) 2015; 31: 121-123. DOI: 10.1051/medsci/20153102002); Patriarca A., Fouillade C. M., Martin F., Pouzoulet F., Nauraye C., et al., Experimental set-up for FLASH proton irradiation of small animals using a clinical system. Int J Radiat Oncol Biol Phys, 102 (2018), pp. 619-626.doi: 10.1016/j.ijrobp.2018.06.403. Epub 2018 Jul. 11).

### Pharmaceutical Composition

The compounds or combinations of the invention may be used or prepared in a pharmaceutical composition.

In some embodiments, the invention relates to a pharmaceutical composition comprising the compounds or combinations of the invention and a pharmaceutical acceptable carrier for use in the treatment of T Cell Acute Lymphoblastic Leukemia (T-ALL) in a subject in need thereof. In some embodiment, the invention relates to a pharmaceutical composition comprising a therapeutically effective amount of an anti-CD3 agent and at least one immunotherapeutic agent, and a pharmaceutical acceptable carrier for use in the treatment of T Cell Acute Lymphoblastic Leukemia (T-ALL).

By a “therapeutically effective amount” of the agent of the present invention as above described is meant a sufficient amount of the agent at a reasonable benefit/risk ratio applicable to any medical treatment. It will be understood, however, that the total daily usage of the agents and compositions of the present invention will be decided by the attending physician within the scope of sound medical judgment. The specific therapeutically effective dose level for any particular patient will depend upon a variety of factors including the disorder being treated and the severity of the disorder; activity of the specific agent employed; the specific composition employed, the age, body weight, general health, sex and diet of the patient; the time of administration, route of administration, and rate of excretion of the specific agent employed; the duration of the treatment; drugs used in combination or coincidental with the specific agent employed; and like factors well known in the medical arts. For example, it is well within the skill of the art to start doses of the agent at levels lower than those required to achieve the desired therapeutic effect and to gradually increase the dosage until the desired effect is achieved. However, the daily dosage of the products may be varied over a wide range from 0.005 to 2,000 mg per adult. Typically, the compositions contain from 0.01 to 1000 mg, in particular from 0.1 to 500 mg of the agent of the present invention for the symptomatic adjustment of the dosage to the patient to be treated. A medicament typically contains from 0.01 mg to 1000 mg of the agent of the present invention, in particular from 0.1 mg to 500 mg of the agent of the present invention, in particular from 1 mg to 500 mg of the agent of the present invention, specifically 25 mg, 50 mg or 400 mg. An effective amount of the drug is ordinarily supplied at a dosage level from 0.005 mg/kg to 20 mg/kg of body weight, especially from 0.01 mg/kg to 10 mg/kg of body weight, especially from 0.1 mg/kg to 1 mg/kg of body weight.

Dosage, mode and frequency of delivery of clinically approved therapeutic mAbs is a subject of intense ongoing research and clinical trials (Hirch I et al., 2022).

According to the invention, the agent of the present invention is administered to the subject in the form of a pharmaceutical composition. Typically, the agent of the present invention may be combined with pharmaceutically acceptable excipients, and optionally sustained-release matrices, such as biodegradable polymers, to form therapeutic compositions. “Pharmaceutically” or “pharmaceutically acceptable” refer to molecular entities and compositions that do not produce an adverse, allergic, or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, an active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal, and intranasal administration forms and rectal administration forms.

Typically, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringeability exists. It must be stable under the conditions of manufacture and storage and must be preserved against the contaminating action of microorganisms, such as bacteria and fungi. Solutions comprising agents of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The agent of the present invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active agents in the required amount in the appropriate solvent with several of the other ingredients enumerated above, as required, followed by filtered sterilization. Generally, dispersions are prepared by incorporating the various sterilized agents of the present inventions into a sterile vehicle which contains the basic dispersion medium and the required other ingredients from those enumerated above. In the case of sterile powders for the preparation of sterile injectable solutions, the typical methods of preparation are vacuum-drying and freeze-drying techniques which yield a powder of the agent of the present invention plus any additional desired ingredient from a previously sterile-filtered solution thereof. The preparation of more, or highly concentrated solutions for direct injection is also contemplated, where the use of DMSO as solvent is envisioned to result in extremely rapid penetration, delivering high concentrations of the active agents to a small tumor area. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

By “therapeutic effect” it is meant an effect induced by an agent or a pharmaceutical composition comprising such agent, capable to prevent or to delay the appearance or development of a disease or disorder, or to cure or to attenuate the effects of a disease or disorder.

In some embodiment, the invention relates to a pharmaceutical composition comprising a therapeutic effective amount of an anti-CD3 agent in combination with an anti-4-1BB agonistic mAb and an anti-OX40 agonistic mAb and a pharmaceutical acceptable carrier for use in the treatment of T Cell Acute Lymphoblastic Leukemia (T-ALL) in a subject in need thereof.

In some embodiment, the invention relates to a pharmaceutical composition comprising a therapeutic effective amount of an anti-CD3 agent in combination with an anti-4-1BB agonistic mAb, an anti-OX40 agonistic mAb and an anti-PD1 antagonistic mAb and a pharmaceutical acceptable carrier for use in the treatment of T Cell Acute Lymphoblastic Leukemia (T-ALL) in a subject in need thereof.

In some embodiment, the invention relates to a pharmaceutical composition comprising a therapeutic effective amount of an anti-CD3 agent in combination with a Fc-fusion protein, in particular etanercept and a pharmaceutical acceptable carrier for use in the treatment of T Cell Acute Lymphoblastic Leukemia (T-ALL) in a subject in need thereof.

In another embodiment, the pharmaceutical composition for use according to the invention comprises a therapeutic effective amount of an anti-CD3 agent in combination with a SMAC mimetic, preferably birinapant.

Every such formulation can also contain other pharmaceutically compatible and nontoxic auxiliary agents, such as, e.g., stabilizers, antioxidants, binders, dyes, emulsifiers, or flavoring substances.

In a particular embodiment, the pharmaceutical composition according to the invention is administered intravenously, orally, by mucosal route, or by nasal route.

Methods for the safe and effective administration are known to those skilled in the art. In addition, administration of anti-cancer agents is described in the standard literature.

In one embodiment, the pharmaceutical composition contains from 0.005 to 2,000 mg of an agent, in particular from 0.01 to 1,000 mg, especially from 0.1 to 500 mg. In a specific embodiment, the pharmaceutical composition contains from 1 mg to 400 mg of an agent, especially 25, 50 or 400 mg.

In one embodiment, the pharmaceutical composition contains an effective amount of the agent at a dosage level from 0.005 mg/kg to 20 mg/kg of body weight, especially from 0.01 mg/kg to 10 mg/kg of body weight, especially from 0.1 mg/kg to 5 mg/kg of body weight, advantageously from 0.1 mg/kg to 1 mg/kg of body weight, advantageously from 0.3 mg/kg to 0.8 mg/kg of body weight.

In one embodiment, the compound, agent, combination and/or pharmaceutical composition of the invention is administered to the patient every week, every two weeks, every three weeks, every four weeks, every five weeks, every six weeks, every seven weeks, or every eight weeks. In particular, every two to six weeks, especially every two or three weeks.

In one embodiment, the compound, agent, combination and/or pharmaceutical composition of the invention is administered to the patient at a rate of one, two, three, four, five, six, seven or eight times per week. In a particular embodiment, the compound, combination and/or pharmaceutical composition of the invention is administered to the patient at a rate of one or two times per week.

In one embodiment, the compound, combination and/or pharmaceutical composition of the invention is administered to the patient for a period of 1 to 12 months, in particular of 1 to 8 months, specially of 2 to 6 months.

The adaptation of posology and administration route for a given patient are common practices well known from those skilled in the art.

The compound, combination agent and/or pharmaceutical composition of the invention may further be administered in addition to other anti-cancer therapy, for example: chemotherapy, radiotherapy, CAR-T cell or CAR-NK cell treatments, immune checkpoint inhibitor treatment and/or therapeutic vaccines.

In one embodiment, said additional active compounds or agent may be contained in the same composition or administrated separately.

In another embodiment, the pharmaceutical composition of the invention relates to combined preparation for simultaneous, separate or sequential use in the treatment of T Cell Acute Lymphoblastic Leukemia (T-ALL) in a subject in need thereof.

The invention also provides kits comprising the compounds of the invention. Kits containing the compounds of the invention find use in therapeutic methods.

The invention will be further illustrated by the following figures and examples. However, these examples and figures should not be interpreted in any way as limiting the scope of the present invention.

### BRIEF DESCRIPTION OF THE FIGURES

FIG. 1: Transcriptome profiling of T-ALL PDX in response to anti-CD3: Leukemic mice were injected with 40μg control IgG, anti-hCD 3 OKT3 or anti-hCD 3 teplizumab for 6 hours. Leukemic cells were then collected, flow-cytometry sorted, RNA isolated and analyzed using human Affymetrix arrays. A Hierarchical clustering on genes deregulated significantly more than 2-fold is shown for IgG 2a, (IgG): teplizumab; (Tepli): OKT3 (OKT3)). B List of genes of interest found significantly upregulated in response to OKT3 and teplizumab. FC: Fold Change. C RNA expression analysis (digitalized numeration of transcripts-mRNA) of genes of interest in M106 xenograft overtime after OKT3 treatment using Nanostring arrays: TNFα, LTα, TNFRSF9 (4-1BB), TNFRSF1B (TNFR2), TNFRSF4 (OX40) and PDCD1 (PD1). D Flow cytometry analysis of TNFRSF1B (TNFR2), TNFRSF1A (TNFR1), TNFRSF9 (4-1BB), TNFRSF4 (OX40) and PDCD1 (PD 1) cell surface expression in leukemic cells isolated from mice treated with 40μg of control IgG or OKT3 for 24h.

FIG. 2: Caspase 8 activity as measured by flow cytometry in primary leukemic cells collected from leukemic mice treated by control IgG (n=3) or anti-CD3 (OKT3) (n=3) mAbs for 6 h.

FIG. 3: A Combination of OKT3 (muromonab-CD3) and urelumab-upper panel: Leukemia burden at time of death of the control arm in NSG mice injected with leukemic cells from a diagnostic T-ALL case (M106) and treated with control IgG, 10 mg/kg of urelumab, 4 μg of OKT3 per mice or combination of OKT3 and urelumab; ns non-significant, *p<0.05; ***p<0.001, ANOVA, Tukey's Multiple Comparison test.—lower panel: Survival curves of NSG mice injected with leukemic cells from a diagnostic T-ALL case and treated with control IgG (control IgG, dotted black line), 4 μg of OKT3 per mice (OKT3, black line), 10 mg/kg of urelumab (urelumab, dotted black line) or combination of OKT3 and urelumab (OKT3 +urelumab, gray line). B Combination of teplizumab and urelumab-upper panel: Leukemia burden at time of death of the control arm in NSG mice injected with leukemic cells from a diagnostic T-ALL case (UPNT 420) and treated with control IgG, 10 mg/kg of urelumab, 4 μg of teplizumab per mice or combination of teplizumab and urelumab; ns non-significant, *p<0.05; ***p<0.001, ANOVA, Tukey's Multiple Comparison test.—lower panel: Survival curves of mice from the same experiment; mice treated with control IgG (control IgG, dotted black line, n=4), mice treated with teplizumab (black line, n=4), mice treated with urelumab (dotted black line; n=4) or combination of teplizumab and urelumab (teplizumab+urelumab, gray line). **p<0.01, log-rank test.

FIG. 4: A Flow cytometry analysis of cell death (PI staining) in control (black symbols) and PD1 knock-down cells (two independents shRNA were used, open and filled circles) in absence (−) or response (+) to anti-CD3 stimulation. Note the increased cell death obtained in response to anti-CD3 stimulation and significant enhancement of cell death in response to anti-CD3 in PD1 knocked-down cells as compared to control cells. B Combination of OKT3 (muromonab-CD3) and nivolumab-upper panel: Leukemia burden at time of death of mice from the control arm in NSG mice injected with leukemic cells from a diagnostic T-ALL case (M106) and treated with control IgG, 4 μg of OKT3 per mice (α-CD3), 10 mg/kg of nivolumab (OPDIVO; α-PD1) or combination of OKT3 and Nivolumab (α-CD3+α-PD1); ns non-significant, **p<0.01; ***p<0.001, ANOVA, Tukey's Multiple Comparison test.−lower panel: Survival curves of mice from the same experiment and treated with control IgG (control IgG, dotted black line), OKT3 (α-CD3, dark grey line) , 10 mg/kg of nivolumab (OPDIVO®; α-PD1 (nivolumab, dotted black line) or combination of OKT3 and nivolumab (OPDIVO®; α-CD3+α-PD1, light grey line). C Combination of OKT3 (muromonab-CD3) and pembrolizumab—upper panel: Leukemia burden at time of death of mice from the control arm in NSG mice injected with leukemic cells from a diagnostic T-ALL case (M106) and treated with control IgG, 4 μg of OKT3 per mice (α-CD3), 10 mg/kg of pembrolizumab (α-PD1) or combination of OKT3 and pembrolizumab (α-CD3+α-PD1); ns non-significant, *p<0.05, ANOVA, Tukey's Multiple Comparison test—lower panel: Survival curves of mice from the same experiment; corresponding treatment is indicated on the curves.

FIG. 5: Survival of NSG mice injected with leukemic cells from a diagnostic T-ALL case and treated with control IgG, 4 μg of OKT3per mice, 20 mg/kg of birinapant (Biri), enbrel (Enbrel, 10 mg/kg,) or combination of either OKT3 and birinapant (OKT3+Biri) or OKT3 and enbrel (OKT3+Enbrel).

FIG. 6: Interest of combining several immunotherapeutic agents with an anti-CD3 agent: Leukemia burden at time of death of mice from the control arm in NSG mice injected with leukemic cells from a diagnostic T-ALL case (M106) and treated with control IgG (Ctr), teplizumab (Tepli, anti-CD3), pembrolizumab (Pembro, PD1 antagonist), urelumab (Ure, 4-1BB agonist) alone or their teplizumab-based combinatory treatment combination according to the invention.

### EXAMPLE

### Materials & Methods

### Mice

NSG female mice (i.e., devoid of T cells, B cells, NK cells; age: 2 months; Charles River Laboratories) were maintained under specific pathogen-free conditions. Experiments were carried out in accordance with the European Union and French National Committee recommendations, under agreement APAFIS #7393-2016102810475144-v1 for the care and use of laboratory animals. Leukemic mice are euthanized when terminally ill, as evidenced by weakness caused by leukemic dissemination in vital organs (bone marrow, lung, and liver).

### Tumor Engraftment, Treatments, and Tumor Expansion Analysis

1×10⁶ fresh leukemic cells obtained from primary NSG mice engrafted with patient-derived cells from T-ALL cases (M106 or UPNT420) are intravenously injected in 2-month-old NSG mice. Tumor load is monitored in blood of engrafted mice using flow cytometry analysis (FSC^{hi}, hCD 7⁺, hCD 45⁺ cells). When tumor load reached 1% of nucleated cells (meaning around 10-20% of leukemic load in BM), mice are injected intravenously with either anti-CD3ε OKT3 mAb (muromonab-CD3, 4 μg/mouse) or the isotype matched (IgG2a) mAb. Similar results were obtained using teplizumab, a humanized, mutated version of OKT3 which is FDA-approved in prevention of type I diabetes (Herold et al., 2019) and with foralumab, a fully human anti-CD3 mAb (Tran Quang et al., 2020). Etanercept (Enbrel) was administered by intraperitoneal route (10 mg/kg) 6 h before anti-CD3 treatments. The c-IAP1/2 inhibitor birinapant (20 mg/kg) was administrated every 3-4 days for 3 weeks. For co-treatments, mice are simultaneously injected once with 4 μg anti-CD 38 OKT3 mAb and 200 μg of either pembrolizumab or nivolumab (anti-PD1 mAbs) or respective control antibodies. Mice are monitored weekly by flow cytometry for leukemic load (FSC^{hi}, hCD7⁺, hCD45⁺ cells) in peripheral blood. Statistical analyses and survival curves are calculated using Prism 5 (GraphPad). One-way ANOVA was performed using Tukey's test and used to analyze leukemia burden. Kaplan-Meier survival curves were compared using the log-rank test. Differences were considered statistically significant at p<0.05 (*), p<0.01 (**) or p<0.001 (***) .

### Flow Cytometry Analysis of Leukemic Cells

Cell suspensions prepared from mouse lymphoid organs, bone marrow, or blood were stained with fluorochrome-labeled antibodies and analyzed on a FACSCanto cytometer (BD Biosciences), as recommended by manufacturer. Data are analyzed using Diva, CellQuest (BD Biosciences) and FlowJo (TreeStar) softwares. Antibodies used are: CD7-FITC (4H9), CD45-APC (2D4) (eBioscience), PD1-Alexa Fluor 647 (EH12.2H7) (Biolegend).

### In Vitro Studies

Jurkat cell line is cultured in RPMI-1640 medium supplemented with 50 μg/ml streptomycin, 50 IU penicillin, and 15% fetal bovine serum (Gibco, Life Technology). Lentivirus are produced as described in Passaro (Passaro et al., 2015) and used to transduce Jurkat cell lines. GFP⁺-transduced cells are sorted using ARIA-FACS sorter. Validation of PD1 knock-down is analyzed by flow cytometry. Apoptosis in response to anti-CD3/CD28 beads (Dynabeads 11131D, Gibco/Thermofisher Scientific) is analyzed using annexin V-APC and propidium iodide (PI) (BD Biosciences).

### Microarray Analysis

Total RNA was isolated from flow-cytometry-sorted leukemic cells (FSChi, hCD7+, hCD 45+) (6 hours following treatment with either 40 μg anti-CD 3 mAb or control IgG 2a antibody), using the Rneasy kit (QIAGEN). cRNA synthesis and hybridization of GeneChip Human Gene 2.0ST Array (Affymetrix) were according to the manufacturer's instructions, as described (http: //www-microarrays. u-strasbg. fr). A paired Student's t-test was performed to compare gene intensities in the different biological replicates. Genes were considered significantly regulated when fold-change was ≥2 and p value≤0.05. Hierarchical clustering analysis and pathway analyses on regulated genes (KEGG) were performed by Genosplice company.

### Validation of Anti-CD3-Induced Signature Using Nanostring Technology

Total RNA was isolated from flow-cytometry-sorted leukemic cells (FSChi, hCD7+, hCD45+) (0, 1,2, 4, 8-, 12-, 24- and 48-hours following treatment with either 40 μg anti-CD 3 mAb or control IgG2a antibody), using the Rneasy kit (QIAGEN). RNA was used with a home-designed panel of capture and reporter probes (Technologie XT custom) and nCounter technology from Nanostring. Panel comprises 95 probes with 5 housekeeping genes and 90 probes that include the most deregulated genes identified in microarray analysis and their related family members.

### Results

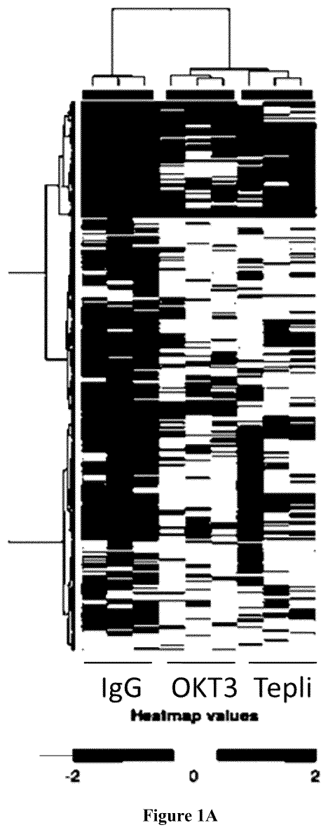

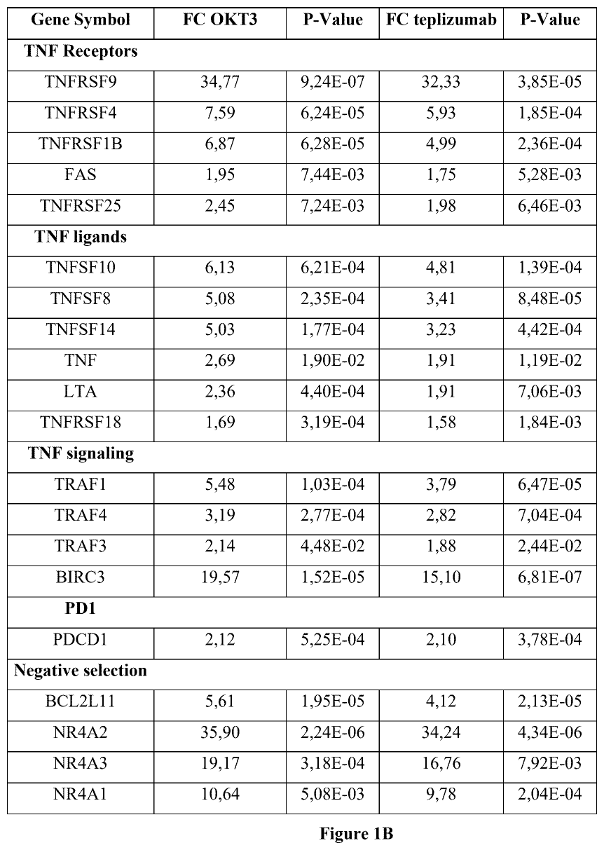

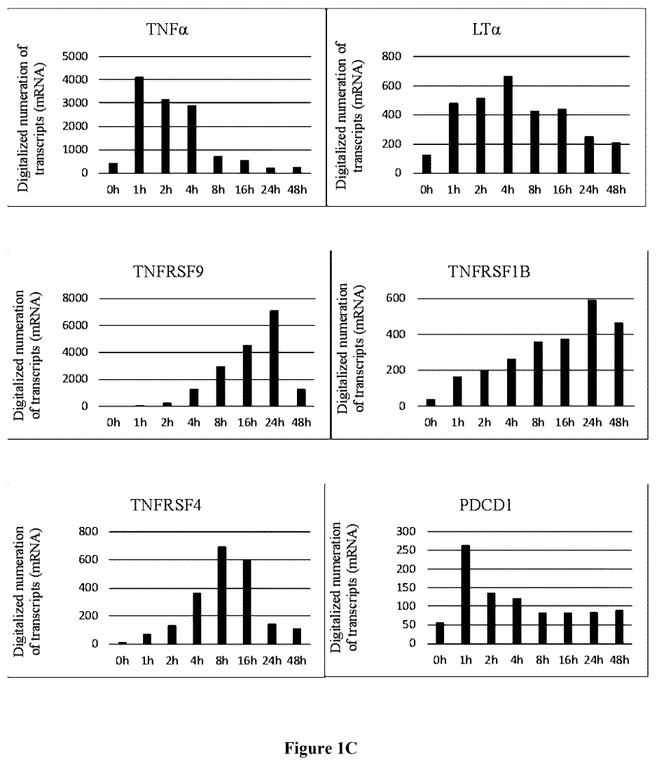

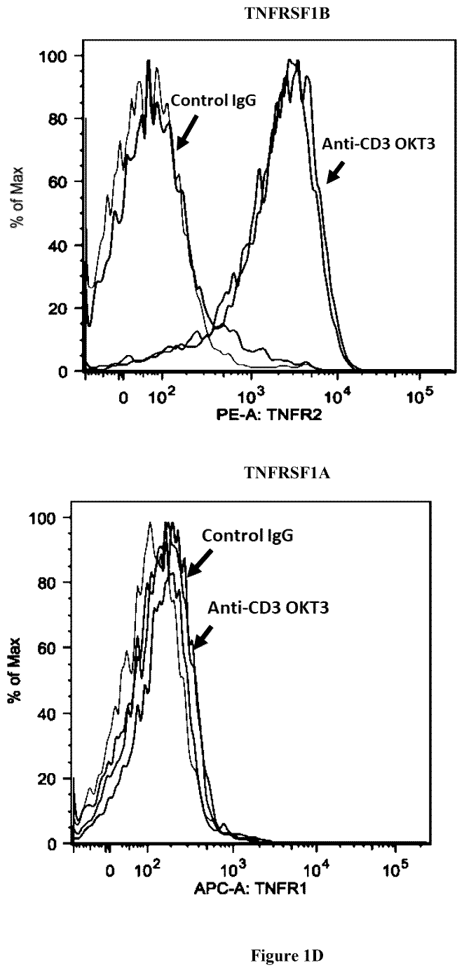

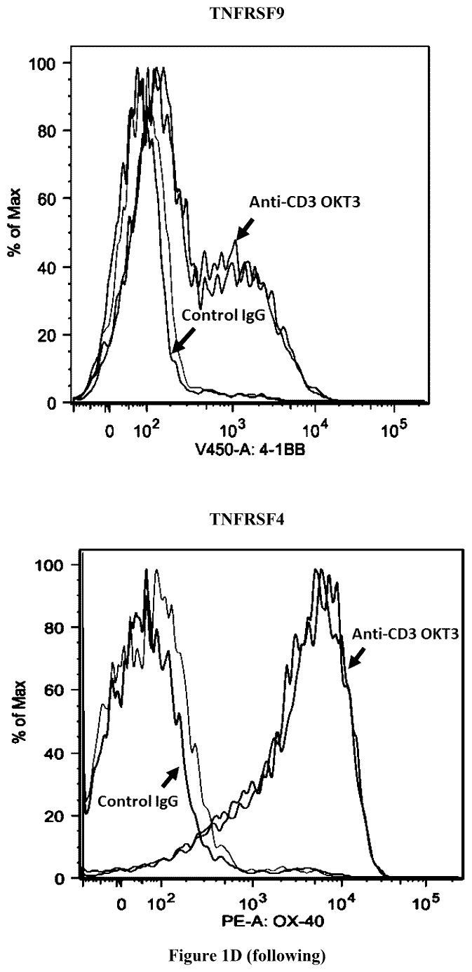

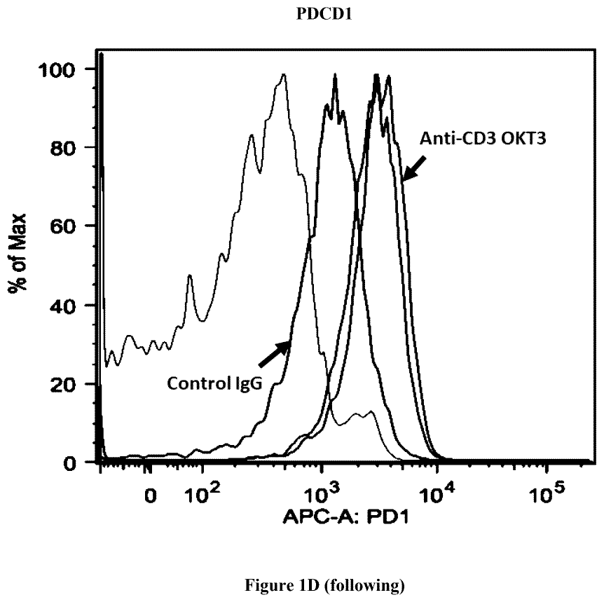

Monotherapy of mice carrying TCR⁺ T-ALL patient derived xenograft (PDX) with anti-CD3 mAbs, while inducing durable remission, ultimately results in leukemia relapse and resistance to TCR-directed therapy (Ghysdeal et al., 2016; Ghysdeal et al., 2020). The inventors thus sought to identify molecular pathways induced by anti-CD3 mAbs in T-ALL cells with the aim to therapeutically target these pathways and create improved vulnerability to anti-CD3 mAbs therapy. To identify pathways instrumental to the anti-leukemic activity of anti-CD3 mAbs, the inventors compared the in vivo global transcriptome induced by Muromonab-CD3 and the clinically relevant anti-CD3 mAb teplizumab in T-ALL PDX cells. The muromonab-CD3/teplizumab samples cluster together and identify a TCR-induced signature in T-ALL of ca 600 deregulated genes (2X; p-value<0.05) common to both monoclonal antibodies (FIG. 1A).

The top signature identified is massive deregulation in ligands (e.g., TRAIL, TNFα, LTα), receptors (e.g., TNFR2, 4-1BB, OX40), adaptors (TRAFs) and effectors (e.g., cIAP2, TNFAIP3/A20) of the TNF signaling pathway as well as the ITIM-containing, inhibitory receptor PD-1 (FIG. 1B). This signature has been validated in PDXs derived from independent T-ALL cases at the RNA and protein levels (FIG. 1C).

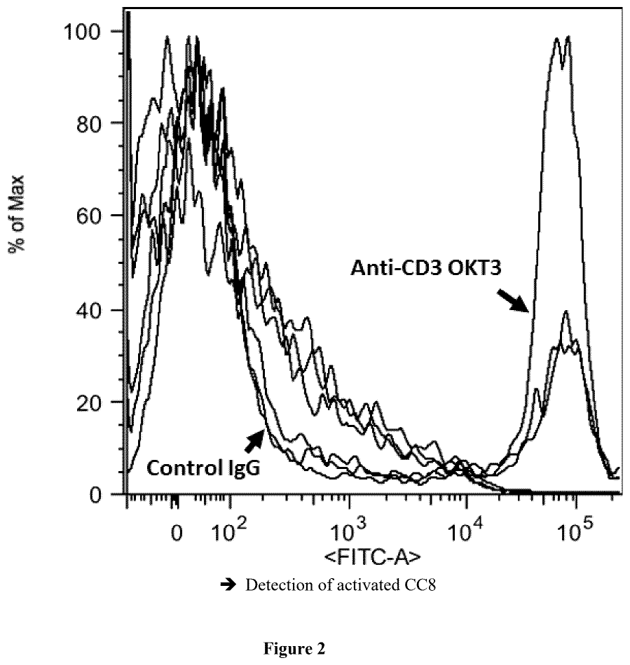

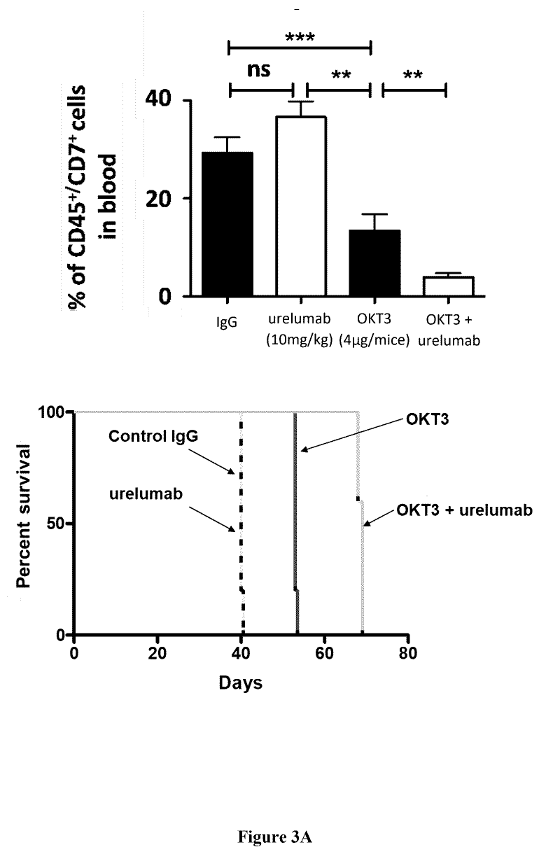

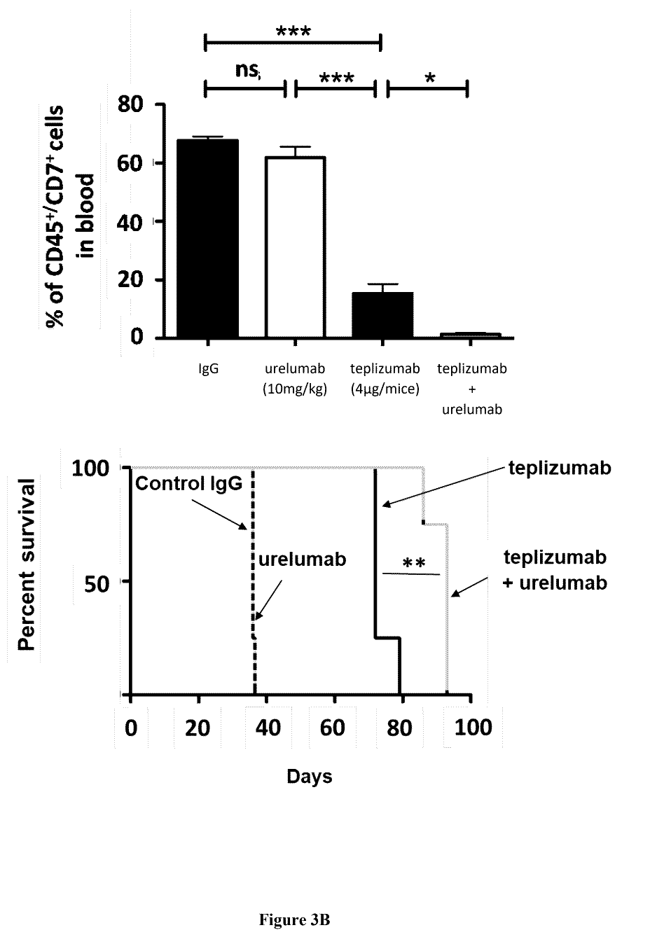

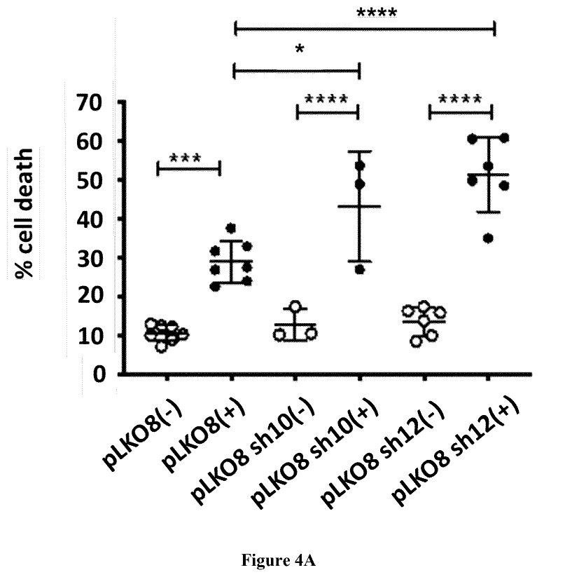

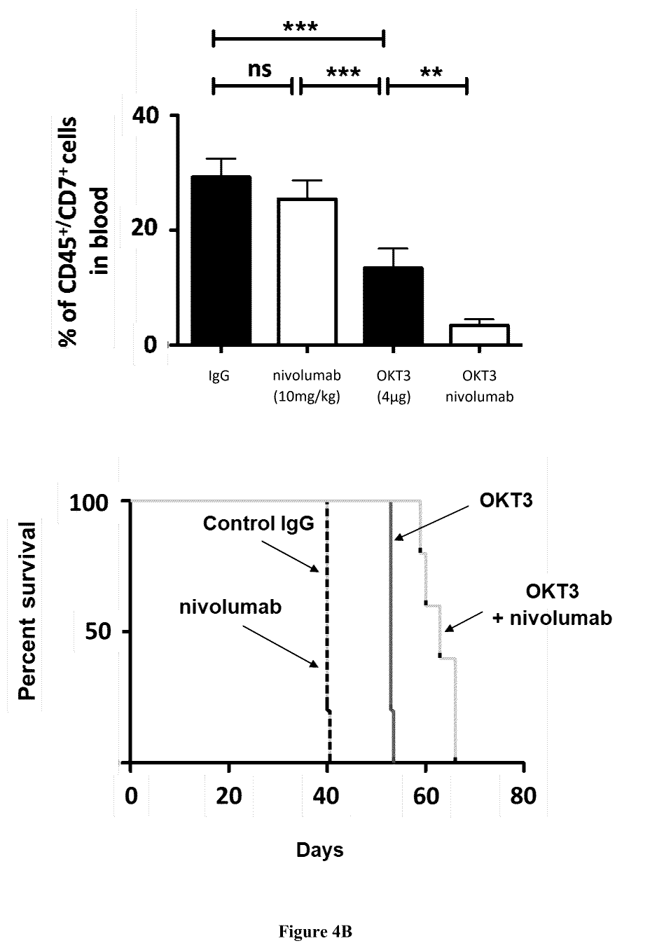

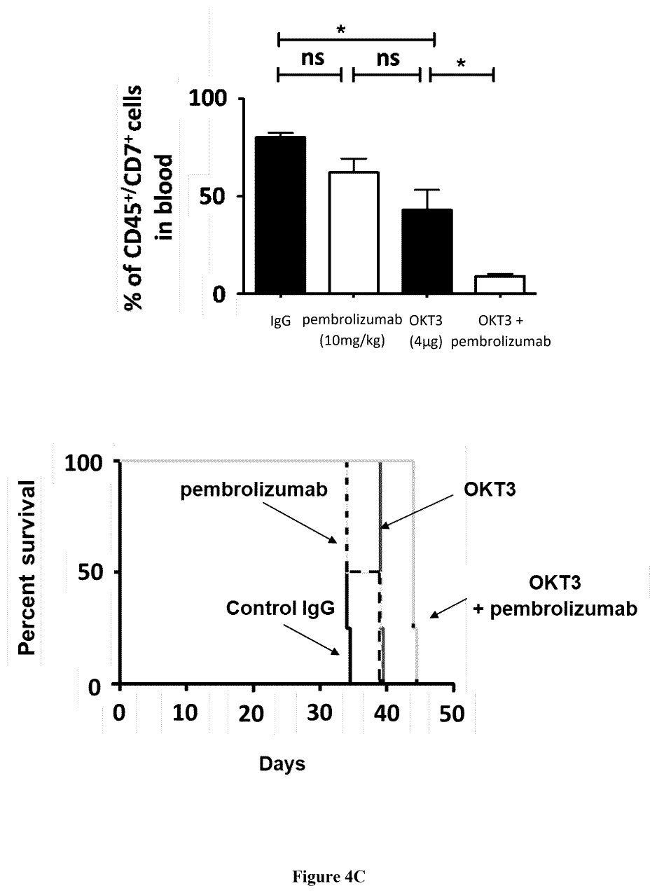

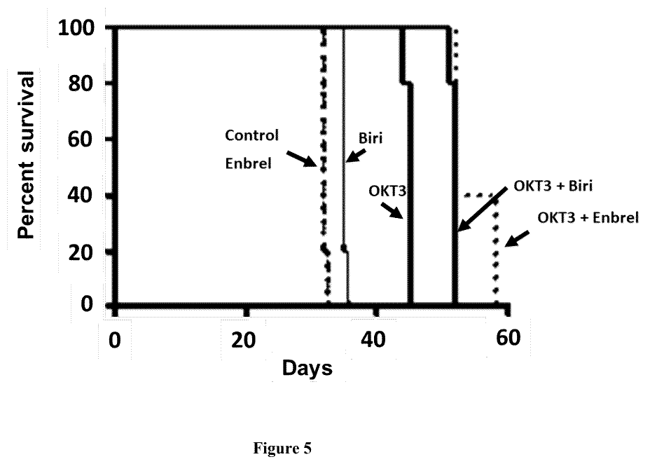

This signature is functionally important as the inventors demonstrate:
(i) fast induction of caspase 8 activity in T-ALL cells in response to OKT3 administration in vivo (FIG. 2).
(ii) in vivo cooperation in anti-leukemic effects between an anti-CD3 mAb (OKT3) and an anti-4-1BB agonistic mAb (urelumab) in T-ALL PDX-bearing NSG mice (FIG. 3A).
(iii) cooperation between anti-CD3 mAb (Dynabeads, Gibco-Thermofisher) and small-hairpin (sh)-mediated knockdown of PD-1 to induce death in a T-ALL cell line in vitro (FIG. 4A).
(iv) in vivo cooperation in anti-leukemic effects between an anti-CD3 mAb (OKT3) and an antagonistic PD-1 mAb (nivolumab-OPDIVO™), an in T-ALL PDX-bearing NSG mice as illustrated by the drop in % of CD45+/CD47+cells and significant increase in survival observed for the animals administered with the combined treatment (FIG. 4B).
(v) in vivo cooperation in anti-leukemic effects between an anti-CD3 mAb (OKT3) and an antagonistic PD-1 mAb (pembrolizumab), an in T-ALL PDX-bearing NSG mice as illustrated by the drop in % of CD45+/CD47+cells and significant increase in survival observed for the animals administered with the combined treatment (FIG. 4C)
(vi) in vivo cooperation in anti-leukemic effects between anti-CD3 mAb and birinapant, an inhibitor of IAP1/2, major effectors in TNFR1/R2 signaling (FIG. 5).
(vii) in vivo cooperation in anti-leukemic effects between anti-CD3 mAb (OKT3) and etanercept (ENBREL) a molecular sink for TNFα/LTα (FIG. 5).
(viii) A synergistic effect between the anti-CD3 agent and the immunotherapeutic agents identified herein by the inventors which target 4-1BB, OX40, PD-1, PDL 1, TRAIL, TNFR1, TNFR2, TNFα/LTα, CD30, CD38, and/or CTLA4.

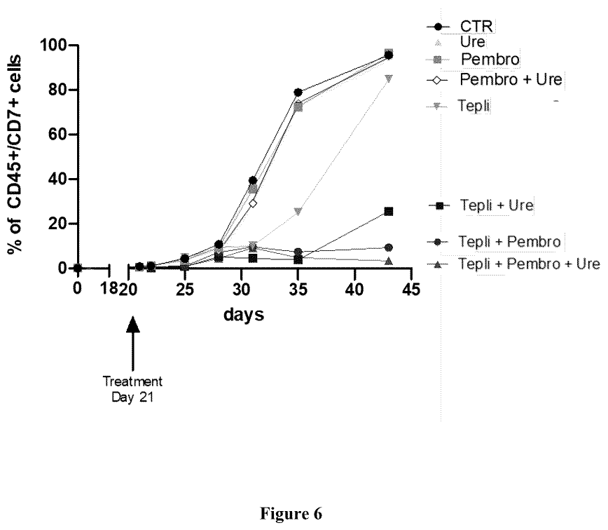

These results in T-ALL PDX in NSG mice (i.e., devoid of T cells, B cells, NK cells) show that clinically relevant anti-4-1BB agonistic mAb, anti-PD1 antagonistic mAb and TNFα/LTαsignaling interference in combination with an anti-CD3 mAb directly target T-ALL cells and improve the therapeutic efficacy of the anti-CD3 therapeutic antibodies (FIG. 3, FIG. 4B, FIG. 4C et FIG. 5). Further, FIG. 6 shows said improvement is further increased when using combined immunotherapeutic agents, e.g. 2 immunotherapeutic agents, one anti-PD1 antagonistic mAb and one 4-1BB agonistic mAb.

### REFERENCES

Throughout this application, various references describe the state of the art to which this invention pertains. The disclosures of these references are hereby incorporated by reference into the present disclosure.
Inaba, H.; Mullighan, C. G., Pediatric acute lymphoblastic leukemia. Haematologica. 105, 2524-2539 (2020).
Girardi, T.; Vicente, C.; Cools, J.; De Keersmaecker, K., The genetics and molecular biology of T-ALL. Blood 129, 1113-1123 (2017)
Trinquand, A.; Dos Santos Nr.; Tran Quang, C.; Rocchetti F.; Zaniboni, B.; Belhocine, M.; Da Costa De Jesus, C.; Lhermitte, L.; Tesio, M.; Dussiot, M.; Cosset, F.; Verhoeyen, E.; Pflumio, F.; Ifrah, N.; Dombret, H.; Spicuglia, S.; Chatenoud, L.; Gross, Da.; Hermine, O.; Macintyre, E.; Asnafi, V.; Ghysdael, J., Triggering the TCR developmental checkpoint activates a therapeutically targetable tumor suppressive pathway in T-cell leukemia. Cancer Discov. 6, 972-985 (2016).
Tran Quang, C.; Zaniboni, B.; Humeau, R.; Lengline, E.; Dourthe, Me.; Ganesan, R.; Singh, S.; Scheer, Jm.; Asnafi, V.; Ghysdael, J., Preclinical Efficacy Of Humanized, Non-Fcyr-Binding Anti-Cd3 Antibodies In T-Cell Acute Lymphoblastic Leukemia. Blood 136, 1298-1302 (2020).
Kuhn, C.; Weiner, H. L., Therapeutic anti-CD3 monoclonal antibodies: from bench to bedside. Immunotherapy 8, 889-906 (2016).
Köhler, G.; Milstein, C., Continuous cultures of fused cells secreting antibody of predefined specificity. Nature 256, 495-497 (1975).
Herold K. C. et al., New England journal of medicine, 2019, Vol381 (7):603-613
Alegre, M. L.; Collins, A. M.; Pulito, V. L.; Brosius, R. A.; Olson, W. C.; Zivin, R. A.; Knowles, R.; Thistlethwaite, J. R.; Jolliffe, L. K.; Bluestone, J. A., Effect of a single amino acid mutation on the activating and immunosuppressive properties of a “humanized” OKT3monoclonal antibody. J. Immunol. 148, 3461-3468 (1992).
Pardoll, D. M., The blockade of immune checkpoints in cancer immunotherapy. Nat. Rev. Cancer. 12, 252-264 (2012).
Bayer, V., An Overview of Monoclonal Antibodies. Semin. Oncol. Nurs. 35, 150927 (2019).
Knaus, H. A.; Kanakry, C. G.; Luznik, L.; Gojo, I., Immunomodulatory Drugs: Immune Checkpoint Agents in Acute Leukemia. Curr. Drug Targets. 18, 315-331 (2017).
Eskiocak, U., et al., Differentiated agonistic antibody targeting CD137 eradicates large tumors without hepatotoxicity. JCI Insight 5, 133647 (2020).
Buchan, S., L., et al., The immunobiology of CD27 and OX40 and their potential as targets for cancer immunotherapy. Blood 131, 39-48 (2018).
Passaro, D.; Irigoyen, M.; Catherinet, C.; Gachet, S.; Da Costa De Jesus, C.; Lasgi, C.; Tran Quang, C.; Ghysdael, J.; CXCR4 Is Required for Leukemia-Initiating Cell Activity in T Cell Acute Lymphoblastic Leukemia. Cancer Cell. 27, 769-79 (2015).
Cordo, V.; van der Zwet, JCG.; Canté-Barrett, K.; Pieters, R.; Meijerink, JPP. T-cell Acute Lymphoblastic Leukemia: A Roadmap to Targeted Therapies. Blood Cancer Discov. 2020 Nov. 24; 2(1):19-31.
Belver, L and AA Ferrando Belver, L. The genetics and mechanisms of T cell acute lymphoblastic leukaemia Nature review Cancer, Vol 16 (8) 494-507 (2016)
Soldevilla, MM, Villanueva H and F Pastor. Aptamers: A Feasible Technology in Cancer Immunotherapy. J. Immunol. Res. Vol 2016: 1083738(2016 )
Jang D et al The Role of Tumor Necrosis Factor Alpha (TNF-2) in Autoimmune Disease and Current TNF-a Inhibitors in Therapeutics. Int. J. Mol. Sci. 22, 2719 (2021)
Javaid, N et al., An orally active, small-molecule TNF inhibitor that disrupts the homotrimerization interface improves inflammatory arthritis in mice. Sci Signal 15, eabi8713 (2022)
Ma BY et al., The expression and the regulatory role of OX 40 and 4-1BB heterodimer in
Chester C, Sanmamed MF, Wang J and I Melero. Immunotherapy targeting 4-1BB: mechanistic rationale, clinical results, and future strategies Blood, 131: 49-57 (2018)
Hirsch I, Goldstein DA, Tannock IF, Butler MO and DC Gilbert. Optimizing the dose and schedule of immune checkpoint inhibitors in cancer to allow global access. Nature Medicine, vol 28: 2236-2237 (2022)
Chodorge M, Züger S, Stirnimann, Briand C, Jermutus L, Grütter MG and RR Minter. A series of Fas receptor agonist antibodies that demonstrate an inverse correlation between affinity and potency. Cell Death Differ. 2012 July; 19(7):1187-95 (2012)
Jafari R, Zolbanin NM, Rafatpanah H, Majidi J, Kazemi T. Fc-fusion Proteins in Therapy: An Updated View. Curr Med Chem. 24(12):1228-1237 (2017)
Menon, A.P.; Moreno, B.; Meraviglia-Crivelli, D.; Nonatelli, F.; Villanueva, H.; Barainka, M.; Zheleva, A.; van Santen, H.M.; Pastor, F. Modulating T Cell Responses by Targeting CD3. Cancers 2023, 15, 1189

1. A method of treating T Cell Acute Lymphoblastic Leukemia (T-ALL) comprising administering to a subject in need thereof an anti-CD3 agent in combination with at least one immunotherapeutic agent.

2. The method according to claim 1, wherein said anti-CD3 agent is a small organic molecule, a polypeptide, an aptamer, an oligonucleotide, or an antiboby.

3. The method according to claim 2, wherein said antibody is a monoclonal antibody (mAb) selected from the group consisting of: foralumab, teplizumab, otelixizumab, visilizumab, blinatumomab, and catumaxomab and combinations thereof.

4. The method according to claim 1, wherein said immunotherapeutic agent is a monoclonal antibody or a Fc-fusion protein.

5. The method according to claim 1, wherein said immunotherapeutic agent is a monoclonal antibody targeting at least one target selected from the group consisting of: PD 1, 4-1BB, OX40, PDL1, TRAIL, TNFR1, TNFR2, TNFα/LTα, CD30, CD38 and/or CTLA4.

6. The method according to claim 1, wherein said immunotherapeutic agent is selected from the group consisting of: an anti-4-1BB agonistic mAb; an anti-OX 40 agonistic mAb; an anti-PD1 antagonistic mAb; an anti-PDL1 antagonistic mAb; anti-TRAIL agonistic mAb; anti-TNFR1 antagonistic mAb; anti-TNFR2 antagonistic mAb; an anti-TNFα/LTα antagonistic mAb; an anti-CD30 mAb; and an anti-CTLA4 mAb, and combinations thereof.

7. The method according to claim 1, further comprising administering to said subject at least two immunotherapeutic agents selected from an anti-4-1BB agonistic mAb, an anti-PD1 antagonistic mAb, an anti-PDL1 antagonistic mAb, an anti-OX40 agonistic mAb, and an anti-TNFα/LTα antagonistic mAb.

8. The method according to claim 7, wherein the combination of at least two immunotherapeutic agents is at least one an anti-4-1BB agonistic mAb and at least one anti-PD1 antagonistic mAb.

9. The method according to claim 7, further comprising administering to said subject an anti-4-1BB agonistic mAb and an anti-OX40 agonistic mAb.

10. The method according to claim 7, wherein the combination of at least two immunotherapeutic agents is at least one anti-4-1BB agonistic mAb, at least one anti-OX40 agonistic mAb and at least one anti-PD1 or anti PDL1 antagonistic mAb.

11. The method according to claim 1, wherein said immunotherapeutic agent is etanercept.

12. A pharmaceutical composition for use in the treatment of T Cell Acute Lymphoblastic Leukemia (T-ALL), comprising a therapeutically effective amount of a anti-CD3 agent, at least one immunotherapeutic agent, and a pharmaceutically acceptable excipient, wherein the anti-CD3 agent is a small organic molecule, a polypeptide, an aptamer, an oligonucleotide, or an antibody, and wherein the immunotherapeutic agent comprises a monoclonal antibody targeting at least one target selected from the group consisting of: PD1, 4-1BB, OX40, PDL1, TRAIL, TNFR1, TNFR2, TNFα/LTα, CD30, CD38 and CTLA4.

13. The pharmaceutical composition according to claim 12, wherein said immunotherapeutic agent is selected from among an anti-4-1BB agonistic mAb, an anti-OX40 agonistic mAb, an anti-PD1 antagonistic mAb, an anti-PDL1 antagonistic mAb and an TNFα/LTα antagonistic mAb, and combinations thereof.

14. The pharmaceutical composition according to claim 12, wherein said immunotherapeutic agent is a combination of at least two immunotherapeutic agents selected from an anti-4-1BB agonistic mAb, an anti-PD1 antagonistic mAb, an anti-PDL1 antagonistic mAb, an anti-OX40 agonistic mAb, and an anti-TNFα/LTα antagonistic mAb.

15. The pharmaceutical composition for use according to claim 14, comprising a therapeutically effective amount of an anti-CD3 in combination with at least one anti-4-1BB agonistic mAb and at least one anti-PD1 antagonistic mAb, wherein said anti-CD3 agent is a small organic molecule, a polypeptide, an aptamer, an oligonucleotide, or an antibody.

16. The pharmaceutical composition according to claim 14, comprising a therapeutically effective amount of an anti-CD3 agent in combination with at least one anti-4-1BB agonistic mAb and at least one anti-OX40 agonistic mAb, wherein said anti-CD3 agent is a small organic molecule, a polypeptide, an aptamer, an oligonucleotide, or an antibody.

17. The pharmaceutical composition according to claim 14, comprising a therapeutically effective amount of an anti-CD3 in combination with at least one anti-4-1BB agonistic mAb, at least one anti-OX40 agonistic mAb and at least one anti-PD1 or anti PDL1 antagonistic mAb, wherein said anti-CD3 agent is a small organic molecule, a polypeptide, an aptamer, an oligonucleotide, or an antibody.

18. The pharmaceutical composition according to claim 14, comprising a therapeutically effective amount of an anti-CD3 agent in combination with an anti-4-1BB agonistic mAb, an anti-OX40 agonistic mAb and an anti-PD1 antagonistic mAb, wherein said anti-CD3 agent is a small organic molecule, a polypeptide, an aptamer, an oligonucleotide, or an antibody.

19. The pharmaceutical composition according to claim 12, wherein said immunotherapeutic agent is a Fc-fusion protein.

20. The method according to claim 1, wherein the administration of said anti-CD3 agent in combination with said at least one immunotherapeutic agent induces cell death of T Cell Acute Lymphoblastic Leukemia (T-ALL) cells in said subject.

21. A pharmaceutical composition for use in the treatment of T Cell Acute Lymphoblastic Leukemia (T-ALL), comprising a therapeutically effective amount of an anti-CD3 agent, at least one immunotherapeutic agent and a pharmaceutically acceptable excipient, wherein said anti-CD3 agent is a monoclonal antibody (mAb) selected from the group consisting of: foralumab, teplizumab, otelixizumab, visilizumab, blinatumomab, and catumaxomab and combinations thereof, and said immunotherapeutic agent is selected from the group consisting of: anti4-1BB agonistic mAb; an anti-OX40 agonistic mAb; an anti-PD1 antagonistic mAb; an anti-PDL1 antagonistic mAb; anti-TRAIL agonistic mAb; anti-TNFR1 antagonistic mAb; anti-TNFR2 antagonistic mAb; an anti-TNFα/LTα antagonistic mAb; an anti-CD30 mAb; and an anti-CTLA4 mAb, and combinations thereof.
